(19) United States
(12) Patent Application Publication
(10) Pub. No.: US 2014/0135327 A1
(43) Pub. Date: May 15, 2014

(54) N- (2 -AMINOPHENYL) BENZAMIDE DERIVATIVES AS HISTONE DEACETYLASE INHIBITORS

(71) Applicants: Ronald Ernest Grigg, Leeds (GB); Martyn Inman, Nottingham (GB); Graham Packham, Salisbury (GB)

(72) Inventors: Ronald Ernest Grigg, Leeds (GB); Martyn Inman, Nottingham (GB); Graham Packham, Salisbury (GB)

(73) Assignee: CANCER RESEARCH TECHNOLOGY LIMITED, London (GB)

(21) Appl. No.: 14/130,703

(22) PCT Filed: Jul. 6, 2012
(86) PCT No.: PCT/GB2012/051594

§ 371 Date: Jan. 3, 2014

(30) Foreign Application Priority Data
Jul. 7, 2011 (GB) 1111630.8

(51) Int. Cl. C07D 413/06; C07D 401/06; C07D 217/14; C07D 217/16; G01N 33/50; C07D 217/24

(52) U.S. Cl. CPC C07D 413/06; G01N 33/5008; C07D 217/24; C07D 217/14; C07D 217/16; C07D 401/06
USPC 514/235.2; 435/7.1; 514/253.05; 514/307; 514/309; 544/128; 544/363; 546/141; 546/145; 546/146; 546/147

(57) ABSTRACT

This invention relates to the discovery of novel compounds, or pharmaceutically acceptable salts thereof, which possess HDAC inhibitory activity. In particular, the compounds of the invention demonstrate selectivity towards Class I HDAC enzymes, and are accordingly expected to be useful for their anti-proliferative activity and in methods of treatment of the human or animal body, for example in preventing or inhibiting tumour growth and metastasis in cancers. The invention also relates to processes for the manufacture of the compounds defined herein, or pharmaceutically acceptable salts thereof, to pharmaceutical compositions containing them and to their use in the manufacture of medicaments for use in the production of an anti-proliferative effect in a warm-blooded animal such as man.

### FIELD OF THE INVENTION

This invention concerns certain novel compounds or pharmaceutically acceptable salts or solvates thereof, processes for preparing these compounds, pharmaceutical compositions comprising them and their use in the treatment, prevention or delay of progression of proliferative disease, such as cancer.

### BACKGROUND OF THE INVENTION

Histone deacetylases (HDACs) are zinc-containing enzymes which catalyse the removal of acetyl groups from the s-amino termini of lysine residues clustered near the amino terminus of nucleosomal histones. There are 18 known HDACs which may be divided into four classes based on their homology to yeast histone deacetylases: Class I (HDACs 1, 2, 3 and 8) which are related to the yeast RPD3 gene; Class II (HDACs 4, 5, 6, 7, 9 and 10) which are related to the yeast Hda1 gene; Class III, also known as the sirtuins, which are related to the Sir2 gene, and Class IV (HDAC11) which contains features of both Class I and II.

Deregulation of certain HDAC inhibitors has been associated with several cancers and HDAC inhibitors such as Trichostatin A have been shown to exhibit significant anti-tumour effects and inhibit cell-growth (Meinke, Current Medicinal Chemistry, 8, 211-235, 2001). According to Yoshida et al (Exper. Cell Res., 177,122-131, 1988), Trichostatin A causes arrest of rat fibroblasts at the G1 and G2 phases of the cell cycle, thereby implicating HDAC in cell cycle regulation. Furthermore, Trichostatin A has been shown to induce terminal differentiation, inhibit cell growth, and prevent the formation of tumours in mice (Finnin et al, Nature, 401, 188-193 (1999)).

Benzamide derivatives having HDAC inhibitory activity are disclosed in WO 2005/121073 and discussed in Zhou et al. J. Med Chem, 2008, 51, 4072-5.

The objective of the present invention is to provide alternative novel compounds that are selective for particular Class I HDAC enzymes, particularly HDAC 2 and/or 3.

### SUMMARY OF THE INVENTION

In accordance with the present invention, the applicants have hereby discovered novel compounds, or pharmaceutically acceptable salts or solvates thereof, which possess HDAC inhibitory activity. In particular, the compounds of the invention demonstrate selectivity towards Class I HDAC enzymes, and are accordingly expected to be useful for their anti-proliferation activity and in methods of treatment of the human or animal body, for example in preventing or inhibiting tumour growth and metastasis in cancers. The compounds of the invention are also expected to be useful agents for the treatment of inflammatory conditions, such as rheumatoid arthritis. The present invention also relates to processes for the manufacture of the compounds defined herein, or pharmaceutically acceptable salts or solvates thereof, to pharmaceutical compositions containing them and to their use in the manufacture of medicaments for use in the production of anti-proliferative/anti-inflammatory activity in warm-blooded animals such as man.

Also, in accordance with the present invention the applicants provide methods of using such compounds, or pharmaceutically acceptable salts thereof, in the treatment of cancer and/or inflammatory conditions such as rheumatoid arthritis.

### DETAILED DESCRIPTION OF THE INVENTION

### Definitions

Unless otherwise stated, the following terms used in the specification and claims have the following meanings:

The term “alkyl” is used herein to refer to a straight or branched chain alkyl moiety. The term “C₁₋₆ alkyl” refers to alkyl groups having 1, 2, 3, 4, 5 or 6 carbon atoms. This term includes groups such as methyl, ethyl, propyl (n-propyl or isopropyl), butyl (n-butyl, sec-butyl or tert-butyl), pentyl, hexyl and the like. Suitably an alkyl group has 1, 2, 3 or 4 carbon atoms. The term alkyl also includes cycloalkyl or cycloalkyl-alkyl groups. For example, the term “C₁₋₆ alkyl” includes C₃₋₆cycloalkyl groups, such as cyclopropyl, cyclobutyl and cyclohexyl and cycloalkyl-alkyl groups such as cyclopropylmethyl. An analogous convention applies to other generic terms, for example “alkenyl” and “alkynyl”.

The term “cycloalkyl” as used herein includes reference to an alicyclic moiety having 3 to 7 carbon atoms. This term includes groups such as cyclopropyl, cyclobutyl, cyclopentyl, cyclohexyl and the like.

The term “alkoxy” as used herein include reference to —O-alkyl groups, wherein the alkyl moiety is a straight or branched chain. The term “C₁₋₆ alkoxy” and comprises 1, 2, 3, 4, 5 or 6 carbon atoms. Suitably, an alkoxy group has 1, 2, 3 or 4 carbon atoms. This term includes reference to groups such as methoxy, ethoxy, propoxy, isopropoxy, butoxy, tert-butoxy, pentoxy, hexoxy and the like.

“Heterocyclyl” is a saturated, unsaturated or partially saturated monocyclic or bicyclic ring containing 4 to 12 atoms of which 1, 2, 3 or 4 ring atoms are chosen from nitrogen, sulphur or oxygen, which ring may be carbon or nitrogen linked, wherein a —CH₂— group can optionally be replaced by a —C(O)—; wherein a ring nitrogen or sulphur atom is optionally oxidised to form the N-oxide or S-oxide(s); wherein a ring —NH is optionally substituted by acetyl, formyl, methyl or mesyl; and wherein a ring is optionally substituted by one or more halo. Suitable examples of the term heterocyclyl include morpholinyl, tetrahydrofuranyl, azetidinyl, pyrrolidinyl, piperidinyl, piperazinyl, and the like.

The term “aryl” means a cyclic or polycyclic aromatic ring system having from 5 to 12 carbon atoms. The term aryl includes both monovalent species and divalent species. Examples of aryl groups include, but are not limited to, phenyl, biphenyl, naphthyl and the like.

The term “heteroaryl” as used herein includes reference to an aromatic heterocyclic ring system having 5 to 12 ring atoms, at least one of which (suitably 1, 2 or 3) is selected from nitrogen, oxygen and sulphur. The group may be a polycyclic ring system, having two or more rings, at least one of which is aromatic, but is more often monocyclic. Illustrative examples of heteroaryls include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, and quinoxalinyl.

The term “halogen” as used herein refers to F, Cl, Br or I.

The term “oxo” is used herein to denote a double bonded oxygen atom. An oxo group is normally attached to a carbon atom so that oxygen atom and carbon together form a carbonyl (—C(O)—) group.

### Compounds

According to a first aspect of the present invention, there is provided a compound of the formula (I):

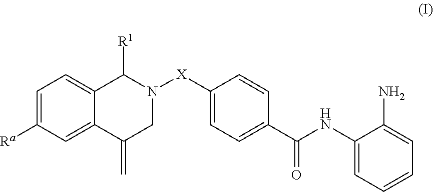

R^{a} is independently selected from hydrogen, —C(O)O—R^{b} or —C(O)NHR^{b}, where R^{b} is hydrogen or a C₁₋₆alkyl group which is optionally substituted by one or more substituents independently selected from halogen, cyano, amino, hydroxy and C₁₋₂alkoxy;
X is —C(O)—, —S—, —S(O)—, —S(O)₂— or a C₁₋₂ alkylene linker;
R¹ is selected from hydrogen, oxo, or a C₁₋₂alkyl group which is optionally substituted by halo, nitro or cyano;
or R¹ is a group:

-Q¹-X¹—R²
wherein:
Q¹ is a C₁₋₂alkylene;
X¹ is selected from —O—, —N(R^{x})—, —C(O)—, —C(O)—O—, —O—C(O)—, —C(O)N(R^{x})—, —N(R^{x})C(O)—;
R^{x} is selected from hydrogen or C₁₋₂alkyl;
R² is selected from hydrogen, C₁₋₄alkyl, phenyl, wherein any C₁₋₄alkyl or phenyl group is optionally substituted with one or more substituents selected from halogen, cyano, amino, hydroxyl, C₁₋₂alkyl, C₁₋₂alkoxy or a group:

X²—R³
where X² is selected from —O—, —NH—, —C(O)—, —C(O)—O—, —O—C(O)—, —C(O)NH—, —NHC(O)—; R³ is C₁₋₄alkyl or C₂₋₄alkenyl which is optionally substituted by halo, hydroxy, amino, cyano, C₁₋₂alkyl, C₁₋₂alkoxy, or a phenyl group which is optionally further substituted by one or more substituent groups selected from hydroxy, halo, cyano, nitro or methoxy;
or R^{x} and R² are linked such that, together with the nitrogen atom to which they are attached, they form a piperidine, piperazine, N-methyl piperazin-4-yl or morpholino ring;
or a pharmaceutically acceptable salt or solvate thereof.

Particular novel compounds of the invention include, for example, compounds of the formula I, or pharmaceutically acceptable salts thereof, wherein, unless otherwise stated, each of R^{a}, X and R¹ has any of the meanings defined hereinbefore or in any of paragraphs (1) to (11) hereinafter:—
(1) R^{a} is independently selected from hydrogen, —C(O)O—R^{b} or —C(O)NHR^{b}, where R^{b} is hydrogen or a C₁₋₄alkyl group which is optionally substituted by one or more substituents independently selected from amino, hydroxy and C₁₋₂alkoxy;
(2) R^{a} is independently selected from hydrogen, —C(O)O—R^{b} or —C(O)NHR^{b}, where R^{b} is hydrogen or a C₁₋₂alkyl group which is optionally substituted by one or more substituents independently selected from amino, hydroxy and C₁₋₂alkoxy;
(3) R^{a} is hydrogen, —C(O)O—CH₃, or —C(O)NH—CH₂—CH₂—NH₂;
(4) X is selected from —S(O)₂— and methylene;
(5) X is methylene;
(6) X is —S(O)₂—;
(7) R¹ is selected from hydrogen, oxo, C₁₋₂alkyl optionally substituted by cyano;
   or R¹ is a group:

-Q¹-X¹—R²
wherein:
   Q¹ is methylene;
X¹ is selected from —O—, —N(R^{x})—, —C(O)—, —C(O)—O—, —O—C(O)—, —C(O)N(R^{x})—, —N(R^{x})C(O)—;
   R^{x} is selected from hydrogen or C₁₋₂alkyl;
   R² is selected from hydrogen, C₁₋₂alkyl, phenyl, wherein any C₁₋₂alkyl or phenyl group is optionally substituted with one or more substituents selected from halogen, cyano, amino, hydroxyl, C₁₋₂alkyl, C₁₋₂alkoxy or a group:

X²—R³
where:
X² is selected from —O—, —NH—, —C(O)—, —C(O)—O—, —O—C(O)—, —C(O)NH—, —NHC(O)—;
R³ is C₁₋₄alkyl or C₂₋₄alkenyl which is optionally substituted by halo, hydroxy, amino, C₁₋₂alkyl, C₁₋₂alkoxy, or a phenyl group which is optionally further substituted by one or more substituent groups selected from hydroxy or methoxy;
or R^{x} and R² are linked such that, together with the nitrogen atom to which they are attached, they form a piperidine, piperazine, N-methyl piperazine or morpholino ring;
(8) R¹ is selected from hydrogen, oxo, C₁₋₂alkyl optionally substituted by cyano;
   or R¹ is a group:

-Q¹-X¹—R²
wherein:
   Q¹ is methylene;
   X¹ is selected from —C(O)—O—, or —C(O)N(R^{x})—;
   R^{x} is hydrogen;
   R² is selected from hydrogen, C₁₋₂alkyl, phenyl, wherein any C₁₋₂alkyl or phenyl group is optionally substituted with one or more substituents selected from halogen, cyano, amino, hydroxyl, or a group:

X²—R³
where:
X² is selected from —C(O)—O— or —C(O)NH—;
R³ is C₁₋₄alkyl or C₂₋₄alkenyl which is optionally substituted by halo, hydroxy, amino, C₁₋₂alkyl, C₁₋₂alkoxy, or a phenyl group which is optionally further substituted by one or more substituent groups selected from hydroxy or methoxy;
or R^{x} and R² are linked such that, together with the nitrogen atom to which they are attached, they form a piperidine, piperazine, N-methyl piperazine or morpholino ring;
(9) R¹ is selected from hydrogen, oxo or methyl optionally substituted by cyano;
   or R¹ is a group:

-Q¹-X¹—R²
wherein:
   Q¹ is methylene;
   X¹ is selected from —C(O)—O—, or —C(O)N(R^{x})—;
   R^{x} is hydrogen;
   R² is selected from hydrogen, C₁₋₂alkyl, phenyl, wherein a C₁₋₂alkyl or phenyl group is optionally substituted with one or more substituents selected from halogen, cyano, amino, hydroxyl, or a group:

X²—R³
where:
X² is selected from —C(O)NH—;
R³ is C₂₋₄alkenyl which is optionally substituted by a phenyl group which is optionally further substituted by one or more substituent groups selected from hydroxy or methoxy;
or R^{x} and R² are linked such that, together with the nitrogen atom to which they are attached, they form a piperidine, piperazine, N-methylpiperazine or morpholino ring;
(10) R¹ is oxo or methyl optionally substituted with cyano;
(11) R¹ is oxo;

In a particular group of compounds of the invention, X is SO₂ or a methylene linker and R^{a} and R¹ have any one of the definitions set out hereinbefore.

In a particular group of compounds of the invention, X is SO₂ or a methylene linker, R^{a} is hydrogen and R¹ has any one of the definitions set out hereinbefore.

Particular examples of compounds of the invention include those shown below. It will of course be appreciated that, where appropriate, each compound may be in the form of the free compound, an acid or base addition salt, or a prodrug.

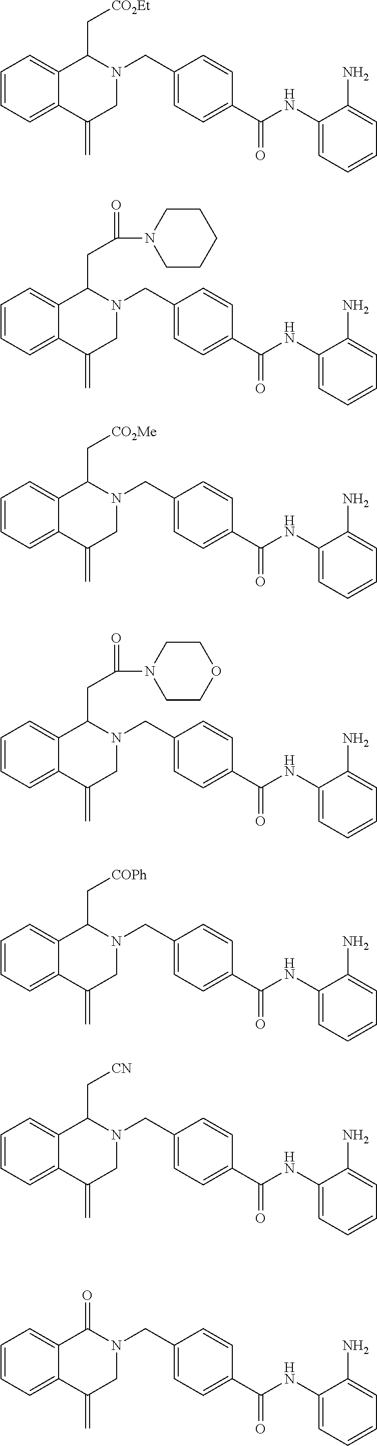

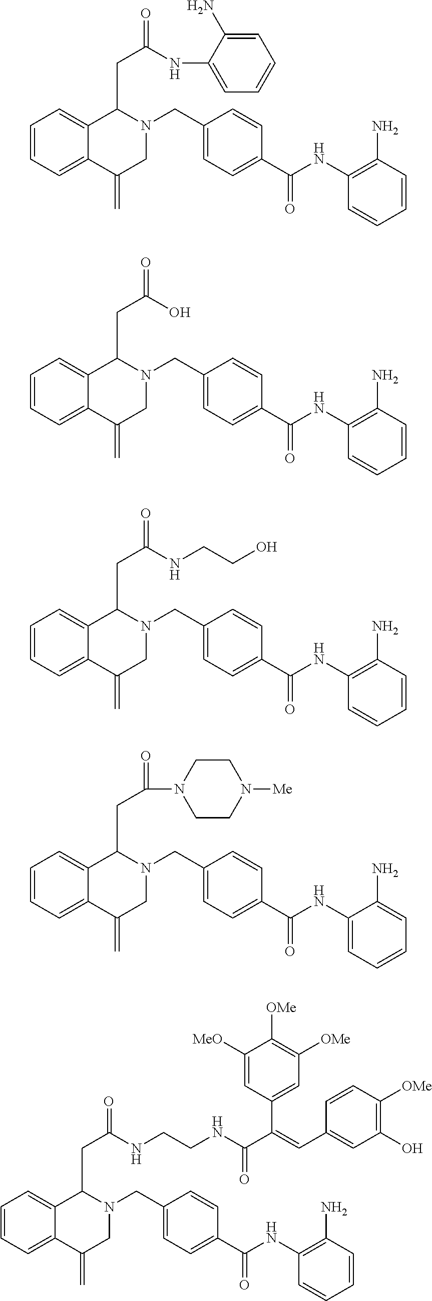 [SMILES: C=C1CN(Cc2ccc(C(=O)Nc3ccccc3N)cc2)C(CC(=O)Nc2ccccc2N)c2ccccc21 ; C=C1CN(Cc2ccc(C(=O)Nc3ccccc3N)cc2)C(CC(=O)O)c2ccccc21 ; C=C1CN(Cc2ccc(C(=O)Nc3ccccc3N)cc2)C(CC(=O)NCCO)c2ccccc21 ; C=C1CN(Cc2ccc(C(=O)Nc3ccccc3N)cc2)C(CC(=O)N2CCN(C)CC2)c2ccccc21 ; C=C1CN(Cc2ccc(C(=O)Nc3ccccc3N)cc2)C(CC(=O)NCCNC(=O)/C(=C/c2ccc(OC)c(O)c2)c2cc(OC)c(OC)c(OC)c2)c2ccccc21]

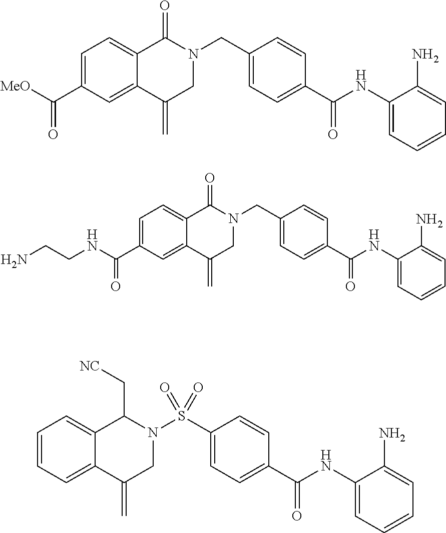 [SMILES: C=C1CN(Cc2ccc(C(=O)Nc3ccccc3N)cc2)C(=O)c2ccc(C(=O)OC)cc21 ; C=C1CN(Cc2ccc(C(=O)Nc3ccccc3N)cc2)C(=O)c2ccc(C(=O)NCCN)cc21 ; C=C1CN(S(=O)(=O)c2ccc(C(=O)Nc3ccccc3N)cc2)C(CC#N)c2ccccc21]

The various functional groups and substituents making up the compounds of the formula I are typically chosen such that the molecular weight of the compound of the formula I does not exceed 1000. More usually, the molecular weight of the compound will be less than 750, for example less than 700, or less than 650, or less than 600, or less than 550. More preferably, the molecular weight is less than 525 and, for example, is 500 or less.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess HDAC inhibitory activity.

It is also to be understood that certain compounds of the formula I may exist in solvated as well as unsolvated forms such as, for example, hydrated forms. It is to be understood that the invention encompasses all such solvated forms that possess HDAC inhibitory activity.

It is also to be understood that certain compounds of the formula I may exhibit polymorphism, and that the invention encompasses all such forms that possess HDAC inhibitory activity.

Compounds of the formula I may exist in a number of different tautomeric forms and references to compounds of the formula I include all such forms. For the avoidance of doubt, where a compound can exist in one of several tautomeric forms and only one is specifically described or shown, all others are nevertheless embraced by formula I. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

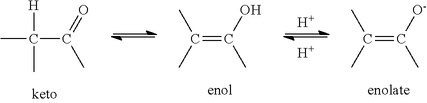

Compounds of the formula I containing an amine function may also form N-oxides. A reference herein to a compound of the formula I that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

The compounds of formula I may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the formula I and in-vivo cleavable amide derivatives that may be formed at a carboxy group or an amino group in a compound of the formula I.

Accordingly, the present invention includes those compounds of the formula I as defined hereinbefore when made available by organic synthesis and when made available within the human or animal body by way of cleavage of a pro-drug thereof. Accordingly, the present invention includes those compounds of the formula I that are produced by organic synthetic means and also such compounds that are produced in the human or animal body by way of metabolism of a precursor compound, that is a compound of the formula I may be a synthetically-produced compound or a metabolically-produced compound.

A suitable pharmaceutically acceptable pro-drug of a compound of the formula I is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14;
h) E. Roche (editor), “Bioreversible Carriers in Drug Design”, Pergamon Press, 1987; and
i) Ferguson et al. Drug Resistance Updates, 4, 225-232 (2001).

A suitable pharmaceutically acceptable pro-drug of a compound of the formula I that possesses a carboxy group is, for example, an in vivo cleavable ester thereof. An in vivo cleavable ester of a compound of the formula I containing a carboxy group is, for example, a pharmaceutically acceptable ester which is cleaved in the human or animal body to produce the parent acid. Suitable pharmaceutically acceptable esters for carboxy include

C₁₋₆alkyl esters such as methyl, ethyl and tert-butyl, C₁₋₆alkoxymethyl esters such as methoxymethyl esters, C₁₋₆alkanoyloxymethyl esters such as pivaloyloxymethyl esters,
3-phthalidyl esters, C₃₋₈cycloalkylcarbonyloxy-C₁₋₆alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆alkoxycarbonyloxy-C₁₋₆alkyl esters such as methoxycarbonyloxymethyl and 1-methoxycarbonyloxyethyl esters.

A suitable pharmaceutically acceptable pro-drug of a compound of the formula I that possesses a hydroxy group is, for example, an in vivo cleavable ester or ether thereof. An in vivo cleavable ester or ether of a compound of the formula I containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁₋₁₀alkanoyl groups such as acetyl, benzoyl, phenylacetyl and substituted benzoyl and phenylacetyl groups, C₁₋₁₀alkoxycarbonyl groups such as ethoxycarbonyl, N,N—(C₁₋₆)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the formula I that possesses a carboxy group is, for example, an in vivo cleavable amide thereof, for example an amide formed with an amine such as ammonia, a C₁₋₄alkylamine such as methylamine, a (C₁₋₄alkyl)₂amine such as dimethylamine, N-ethyl-N-methylamine or diethylamine, a C₁₋₄alkoxy-C₂₋₄alkylamine such as 2-methoxyethylamine, a phenyl-C₁₋₄alkylamine such as benzylamine and amino acids such as glycine or an ester thereof.

A suitable pharmaceutically acceptable pro-drug of a compound of the formula I that possesses an amino group is, for example, an in vivo cleavable amide derivative thereof. Suitable pharmaceutically acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl.

The in vivo effects of a compound of the formula I may be exerted in part by one or more metabolites that are formed within the human or animal body after administration of a compound of the formula I. As stated hereinbefore, the in vivo effects of a compound of the formula I may also be exerted by way of metabolism of a precursor compound (a pro-drug).

### Synthesis

In a further aspect, the present invention relates to a process for preparing a compound of the invention as defined herein.

By way of illustration, compounds of the invention may be synthesised according to the procedures given herein. It will be understood that these processes are solely for the purpose of illustrating the invention and should not be construed as limiting. A process utilising similar or analogous reagents and/or conditions known to one skilled in the art may also be used to obtain a compound of the invention.

Any mixtures of final products or intermediates obtained can be separated on the basis of the physico-chemical differences of the constituents, in a known manner, into the pure final products or intermediates, for example by chromatography, distillation, fractional crystallisation, or by the formation of a salt if appropriate or possible under the circumstances.

### Pharmaceutical Compositions

The compounds of the invention will normally be administered orally, intravenously, subcutaneously, buccally, rectally, dermally, nasally, tracheally, bronchially, by any other parenteral route, as an oral or nasal spray or via inhalation, The compounds may be administered in the form of pharmaceutical preparations comprising prodrug or active compound either as a free compound or, for example, a pharmaceutically acceptable non-toxic organic or inorganic acid or base addition salt, in a pharmaceutically acceptable dosage form. Depending upon the disorder and patient to be treated and the route of administration, the compositions may be administered at varying doses.

Typically, therefore, the pharmaceutical compounds of the invention may be administered orally or parenterally (“parenterally” as used herein, refers to modes of administration which include intravenous, intramuscular, intraperitoneal, intrasternal, subcutaneous and intraarticular injection and infusion) to a host. In the case of larger animals, such as humans, the compounds may be administered alone or as compositions in combination with pharmaceutically acceptable diluents, excipients or carriers.

Actual dosage levels of active ingredients in the pharmaceutical compositions of this invention may be varied so as to obtain an amount of the active compound(s) that is effective to achieve the desired therapeutic response for a particular patient, compositions, and mode of administration. The selected dosage level will depend upon the activity of the particular compound, the route of administration, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

In certain embodiments, an appropriate dosage level will generally be about 0.01 to 500 mg per kg patient body weight per day which can be administered in single or multiple doses. In a particular embodiment, the dosage level is about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions may be provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0 and 1000.0 milligrams of the active ingredient for the symptomatic adjustment of the dosage to the patient to be treated. The compounds may be administered on a regimen of 1 to 4 times per day, e.g. once or twice per day. The dosage regimen may be adjusted to provide the optimal therapeutic response.

According to a further aspect of the invention there is thus provided a pharmaceutical composition including a compound of the invention as defined herein, in admixture with a pharmaceutically acceptable adjuvant, diluent or carrier.

Pharmaceutical compositions of this invention for parenteral injection suitably comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants.

These compositions may also contain adjuvants such as preservative, wetting agents, emulsifying agents and dispersing agents. Prevention of the action of microorganisms may be ensured by the inclusion of various antibacterial and antifungal agents, for example, paraben, chlorobutanol or phenol sorbic acid. It may also be desirable to include isotonic agents such as sugars or sodium chloride, for example. Prolonged absorption of the injectable pharmaceutical form may be brought about by the inclusion of agents (for example aluminum monostearate and gelatin) which delay absorption.

Solid dosage forms for oral administration include capsules, tablets, pills, powders and granules. In such solid dosage forms, the active compound is typically mixed with at least one inert, pharmaceutically acceptable excipient or carrier such as sodium citrate or dicalcium phosphate and/or one or more: a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

Suitably, oral formulations contain a dissolution aid. The dissolution aid is not limited as to its identity so long as it is pharmaceutically acceptable. Examples include nonionic surface active agents, such as sucrose fatty acid esters, glycerol fatty acid esters, sorbitan fatty acid esters (e.g. sorbitan trioleate), polyethylene glycol, polyoxyethylene hydrogenated castor oil, polyoxyethylene sorbitan fatty acid esters, polyoxyethylene alkyl ethers, methoxypolyoxyethylene alkyl ethers, polyoxyethylene alkylphenyl ethers, polyethylene glycol fatty acid esters, polyoxyethylene alkylamines, polyoxyethylene alkyl thioethers, polyoxyethylene polyoxypropylene copolymers, polyoxyethylene glycerol fatty acid esters, pentaerythritol fatty acid esters, propylene glycol monofatty acid esters, polyoxyethylene propylene glycol monofatty acid esters, polyoxyethylene sorbitol fatty acid esters, fatty acid alkylolamides, and alkylamine oxides; bile acid and salts thereof (e.g. chenodeoxycholic acid, cholic acid, deoxycholic acid, dehydrocholic acid and salts thereof, and glycine or taurine conjugate thereof); ionic surface active agents, such as sodium laurylsulfate, fatty acid soaps, alkylsulfonates, alkylphosphates, ether phosphates, fatty acid salts of basic amino acids; triethanolamine soap, and alkyl quaternary ammonium salts; and amphoteric surface active agents, such as betaines and aminocarboxylic acid salts.

The solid dosage forms of tablets, dragees, capsules, pills, and granules can be prepared with coatings and shells such as enteric coatings and other coatings well known in the pharmaceutical formulating art. They may optionally contain opacifying agents and may also be of a composition such that they release the active ingredient(s) only, or preferentially, in a certain part of the intestinal tract, and/or in delayed fashion. Examples of embedding compositions include polymeric substances and waxes.

The active compounds may also be in micro-encapsulated form, if appropriate, with one or more of the above-mentioned excipients.

The active compounds may be in finely divided form, for example they may be micronised.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents. Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar, and tragacanth and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolisable lipid capable of forming liposomes can be used. The present compositions in liposome form can contain, in addition to a compound of the present invention, stabilisers, preservatives, excipients and the like. The preferred lipids are the phospholipids and the phosphatidyl cholines (lecithins), both natural and synthetic. Methods to form liposomes are known in the art, for example, Prescott, Ed., Methods in Cell Biology, Volume XIV, Academic Press, New York, N.Y. (1976), p 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants which may be required. Ophthalmic formulations, eye ointments, powders and solutions are also contemplated as being within the scope of this invention.

### Therapeutic Uses

Compounds of the invention may be useful in the therapy of a variety of diseases and conditions. The subject of said therapy may be a human or an animal. Compounds of the invention may exhibit desirable potency, selectivity and microsomal stability. The compounds may be useful in the therapy of diseases or conditions in which HDACs are implicated.

In particular, compounds of the invention may be useful in the treatment or prevention of cancer and/or inflammatory conditions such as rheumatoid arthritis.

### Cancer Treatment

In a particular embodiment of the invention, the compounds of the invention are for use in the treatment of cancer.

The term “cancer” as used herein includes reference to cellular proliferative or differentiative disorders, including all types of cancerous growths or oncogenic processes, metastatic tissues or malignantly transformed cells, tissues, or organs, irrespective of histopathologic type or stage of invasiveness. Included are malignancies of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. This term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

The compounds of the invention may also be useful in the treatment of a variety of proliferative disorders. Such disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. The diseases may arise from poorly differentiated acute leukemias, e.g. erythroblastic leukemia and acute megakaryoblastic leukemia. Additional exemplary myeloid disorders include acute promyeloid leukemia, acute myelogenous leukemia and chronic myelogenous leukemia; lymphoid malignancies such as acute lymphoblastic leukemias, including B-lineage and T-lineage types, chronic lymphocytic leukemia, prolymphocytic leukemia, hairy cell leukemia and Waldenstrom's macroglobulineniia. Additional forms of malignant lymphomas include non-Hodgkin lymphoma and variants thereof, peripheral T cell lymphomas, adult T cell leukemia/lymphoma, cutaneous T-cell lymphoma, large granular lymphocytic leukemia, Hodgkin's disease and Reed-Sternberg disease. In particular, the compounds may be used in the therapy of haematologic cancers such as leukaemia, non-small cell lung cancers, colonic cancers, breast cancers, ovarian cancers, renal cancers, melanoma, carcinoma, sarcoma and metastatic disorders.

Examples of cancerous disorders of the colon include, but are not limited to, non-neoplastic polyps, adenomas, familial syndromes, colorectal carcinogenesis, colorectal carcinoma, and carcinoid tumors.

Examples of cancerous disorders of the liver include, but are not limited to, nodular hyperplasias, adenomas, and malignant tumors, including primary carcinoma of the liver and metastatic tumors.

Examples of cancerous disorders of the ovary include, but are not limited to, ovarian tumors such as, tumors of coelomic epithelium, serous tumors, mucinous tumors, endometeriod tumors, clear cell adenocarcinoma, cystadenofibroma, brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecoma-fibromas, androblastomas, hill cell tumors, and gonadoblastoma; and metastatic tumors such as Krukenberg tumors.

Examples of cancerous disorders of the breast include, but are not limited to, proliferative breast disease including, e.g. epithelial hyperplasia, sclerosing adenosis, and small duct papillomas; tumors, e.g. stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular ‘carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

In particular, the compounds may be useful in the therapy of a disorder selected from leukaemia, colonic cancer, melanoma and non-small cell lung cancer.

The Particular Role of HDAC-3 Inhibition There are 3 distinct classes of Zn-dependent HDACs targeted by HDAC inhibitors currently in clinical development; class I (HDACs 1, 2, 3 and 8), class II (HDACs 4, 5, 6, 7, 9 and 10) and class IV (HDAC11)¹. In contrast to class II/IV enzymes, class I enzymes (and HDACs1-3 in particular) are widely expressed and are predominantly localized to the nucleus where they play key roles in suppressing gene expression in both normal and malignant cells. Whereas HDAC 1 and HDAC2 participate in various co-repressor complexes, HDAC3 is the only HDAC which is a “core” component of the closely related N-CoR and SMRT repressor complexes². Additional HDACs have been shown to be associated with these corepressor complexes, but only HDAC3 is an essential component of stable (i.e. salt and detergent resistant) N-CoR/SMRT complexes. N-CoR/SMRT are also essential for HDAC3 activity^{3,4}. Therefore, HDAC3 activity is tightly linked to N-CoR/SMRT and HDAC3 selective inhibitors are therefore expected to have therapeutic activity in cancers in which N-CoR/SMRT-mediated transcriptional dysregulation contributes to malignant development and progression.

In acute myeloid leukaemia (AML), N-CoR/SMRT complexes have been unequivocally linked to leukaemogenesis. AML is the most common acute leukaemia in adults, with approximately 2,200 new cases diagnosed and 2,100 deaths in the UK each year. The incidence of AML is expected to rise with an aging population and increased occurrence of therapy-related AML in previously treated cancer survivors.

The hallmark of AML is a block of normal cellular differentiation⁵. N-CoR/SMRT corepressor complexes play a central role in this differentiation block via transcriptional repression of differentiation promoting genes. For example, about 40% of AML contain recurrent chromosomal translocations giving rise to fusion proteins that drive transcriptional repression of genes. N-CoR/SMRT complexes have been shown play a central role in the suppression of gene expression mediated by the PML-RARα, AML1-ETO and CBFβ-MYH11 fusion proteins which, together, account for approximately one-third of AML cases⁶⁻¹¹. Importantly, siRNA-mediated knock-down of HDAC3 is sufficient to block the transcriptional repressing activity of PML-RARα¹² directly validating HDAC3 inhibition as a strategy to overcome N-CoR/SMRT complex mediated transcriptional repression (FIG. 1). All-trans retinoic acid (ATRA) is used a therapeutic agent in a specific subset of AML (acute promyelocytic leukaemia, APL) where it reverses retinoic acid receptor a (RARα)-mediated transcriptional repression, leading to growth inhibition, differentiation and apoptosis¹³. However, the majority of AML do not respond to ATRA and, in APL, de novo and acquired resistance are significant clinical problems. By targeting HDAC3, a central component of the transcriptional repression machinery, selective HDAC inhibitors offer a novel approach to “differentiation” therapy for AML.

The potential to target N-CoR/SMRT corepressor complexes via HDAC inhibitors in AML is clearly demonstrated using non-selective inhibitors. Non-selective HDAC inhibitors activate RARα-dependent gene expression and promote differentiation in AML cells, even in leukaemic cells which are resistant to ATRA^{7,14}. HDAC inhibitors appear to have clinical activity in APL¹⁵, however, current HDAC inhibitors have not been optimised for selective activity against HDAC3.

In addition to AML, N-CoR/SMRT complexes have also been implicated in a number of other malignancies (including diffuse large B-cell lymphoma and some types of breast cancer) and siRNA-mediated knock-down of HDAC3 is sufficient to decrease proliferation, survival and/or migration in ovarian, colon, cervical and synovial carcinoma cell lines¹⁶⁻²². Thus, selective inhibition of HDAC3 is likely to be an effective strategy for the treatment of various cancer types, including more common epithelial malignancies

The compounds of the present invention show selectivity towards HDAC-3. Accordingly, these compounds are considered to be potentially useful therapeutic agents for the treatment of malignant disease in which N-CoR/SMT complexes are implicated, such as AML, diffuse large B-cell lymphoma, some types of breast cancer, as well as ovarian, colon, cervical and synovial cancers.

In a particular embodiment, the compounds of the invention are for use in the treatment of AML.

### Inflammation

In a further embodiment, the compounds of the invention are for use in treatment inflammatory conditions, such as rheumatoid arthritis. There is increasing evidence becoming available in the literature to suggest that HDAC inhibitors are potentially useful agents for the treatment of inflammatory conditions such as rheumatoid arthritis (see, for example, Choo et al. Histone deacetylase inhibitors: new hope for rheumatoid arthritis? Curr. Pharm. Des 2008; 14(8):803-820; Halili et al. Histone deacetylase inhibitors in inflammatory disease. Curr Top Med Chem 2009; 9(3):309-319; Lin et al. Anti-rheumatic activities of histone deacetylase (HDAC) inhibitors in vivo in collagen-induced arthritis in rodents. Br. J. Pharmacol 2007; 150(7):862-872; and Grabiec et al. Histone deacetylase inhibitors suppress inflammatory activation of rheumatoid arthritis patient synovial macrophages and tissue. J. Immunol 2010; 184(5):2718-2728).

### FIGURES

FIG. 1 shows a diagram showing the HDAC3-mediated transcriptional repression in APL. The AML-associated PML-RARα fusion protein silences expression of genes normally required for myeloid cell differentiation by binding to retinoic acid response elements (RARE) in the promoter of RARα target genes and recruitment of SMRT/N-CoR corepressor complexes. In addition to HDAC3, the other “core” components of SMRT/N-CoR corepressor complexes are GPS2, TBL1 and TBL1R. Recruitment of HDAC3 leads to repression of target gene transcription Inhibition of HDAC3 activity (by small chemical compounds or siRNA) prevents PML-RARα-mediated repression leading to induction of RARα target gene expression and results in differentiation, cell cycle arrest and/or apoptosis in leukaemic cells. The model is based on PML-RARα positive APL but N-CoR/SMRT complexes are also involved in transcriptional repression mediated by other leukaemic fusion proteins and BCL-6 (in this case the complex is termed B-CoR).

FIG. 2(i) shows the growth inhibition in HL60 and NB4 cells (4 day, MTS assay) as described in Example 18.

FIG. 2(ii) shows the differentiation in HL60 cells (3 days, CD11b flow cytometry) as described in Example 18.

FIG. 2(iii) shows the CYP26RNA expression in HL60 (left) and NB4 (right) cells (1 day assay, Q-RT-PCR) as described in Example 18.

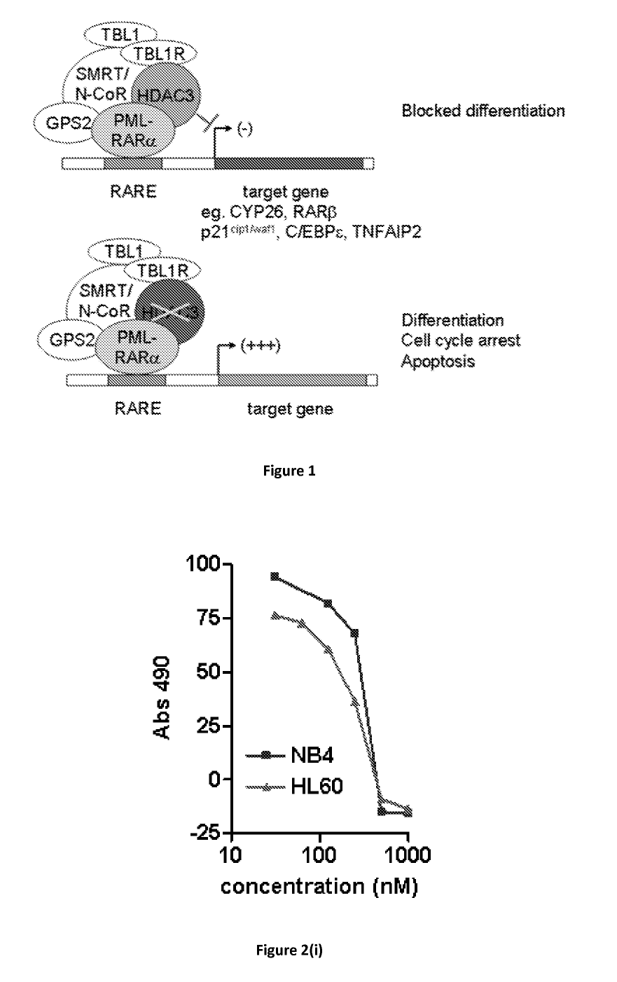

### EXAMPLES

The following Examples illustrate the invention. The numbering given in the structures refers to the location of atoms as identified by NMR.

### General Procedure for 4-methylene-1,2,3,4-tetrahydroisoquinoline formation

Aryl iodide (1.0 mol equiv), nucleophile (1.1 mol equiv), Pd₂dba₃ (0.025 mol equiv), tri-2-furylphosphine (0.05 mol equiv), K₂CO₃ (2.0 mol equiv) and DMF (10-20 ml/mmol) were combined in a Schlenk tube with a stirrer bar, subjected to two freeze-pump-thaw cycles and charged with allene (0.5 bar). The Schlenk tube was thawed and stirred vigorously at 80° C. for 24 h, cooled to room temperature, diluted with H₂O (60 ml) and extracted with EtOAc (2×20 ml). The combined organic extracts were washed with H₂O (20 ml), dried (MgSO₄), filtered and concentrated in vacuo. Column chromatography of the residue afforded the product.

### Intermediate 1: 1-[(2E)-3-(2-Iudophenyl)prop-2-enoyl]-4-methylpiperazine

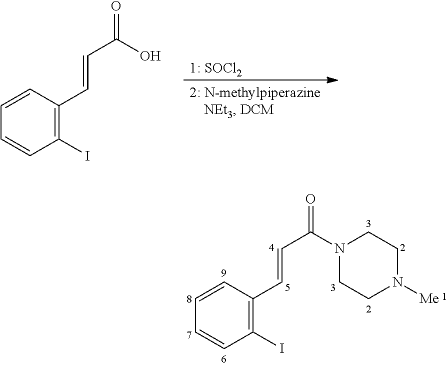 [SMILES: O=C(O)/C=C/c1ccccc1I ; CN1CCN(C(=O)/C=C/c2ccccc2I)CC1]

2-Iodocinnamic acid (1.37 g, 5 mmol) and SOCl₂ (6 mL) were stirred at reflux for 2 h, and the excess SOCl₂ removed in vacuo. DCM (2×10 mL) was used to azeotrope residual traces of SOCl₂. The solid residue was dissolved in DCM (10 mL), and N-methylpiperazine (1.00 g, 10 mmol) and NEt₃ (2.02 g, 20 mmol) added successively, dropwise over 1 min each at 0° C. with stirring. The resulting mixture was stirred for 18 h, washed successively with HCl (1M aq., 10 mL), NaHCO₃ (sat. aq., 10 mL) and brine (10 mL), dried (MgSO₄), filtered and concentrated in vacuo. The residue was triturated with hexane to give the product as pale yellow needles (1.20 g, 67%), m.p. 120-122° C.

δH (CDCl₃, 500 MHz): 7.89 (d, J 7.8, 1H, H-9), 7.78 (d, J 15.3, 1H, H-5), 7.52 (dd, J 7.5, 1.4, 1H, H-6), 7.34 (t, J 7.5, 1H, H-7), 7.02 (ddd, J 7.8, 7.5, 1.4, 1H, H-8), 6.70 (d, J 15.3, 1H, H-4), 3.77 (s, b, 2H, H-2), 3.67 (s, b, 2H, H-2), 2.45 (t, J 5.1, 4H, H-3), 2.33 (s, 3H, H-1).

δC (CDCl₃, 75 MHz): 164.9, 145.6, 139.9, 138.9, 130.6, 128.4, 127.2, 120.7, 100.7, 54.7, 46.0, 42.1.

v/max (film) 2939, 2797, 1643, 1603, 1463.

### Intermediate 2: 4-[(2E)-3-(2-Iodophenyl)prop-2-enoyl]morpoline

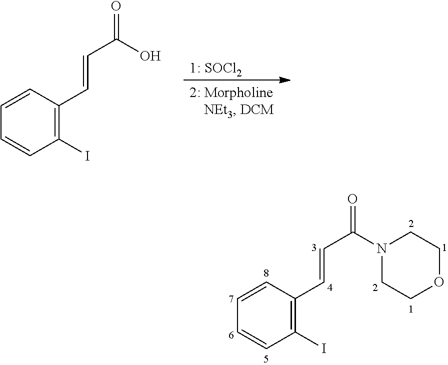 [SMILES: O=C(O)/C=C/c1ccccc1I ; O=C(/C=C/c1ccccc1I)N1CCOCC1]

Prepared in a similar manner to Intermediate 1 using 2-iodocinnamic acid (1.37 g) and morpholine (0.87 g). The product was obtained as pale yellow needles (1.18 g, 71%), m.p. 100-102° C.

δH (CDCl₃, 500 MHz): 7.89 (d, J 7.8, 1H, H-8), 7.81 (d, J 15.3, 1H, H-4), 7.52 (dd, J 7.7, 1.4, 1H, H-5), 7.35 (dd, J 7.7, 7.6, 1H, H-6), 7.03 (ddd, J 7.8, 7.6, 1.4, 1H, H-7), 6.68 (d, J 15.3, 1H, H-3), 3.74-3.67 (m, 8H, H-1 & H-2).

δC (CDCl₃, 75 MHz): 165.5, 146.5, 140.4, 139.2, 131.1, 128.9, 127.6, 120.5, 101.2, 67.3, 46.8, 42.9.

v/max (film) 3057, 2856, 1645, 1604, 1463.

### Intermediate 3: Tert-Butyl (4-{[(2-aminophenyl)amino]carbonyl}benzyl)carbamate

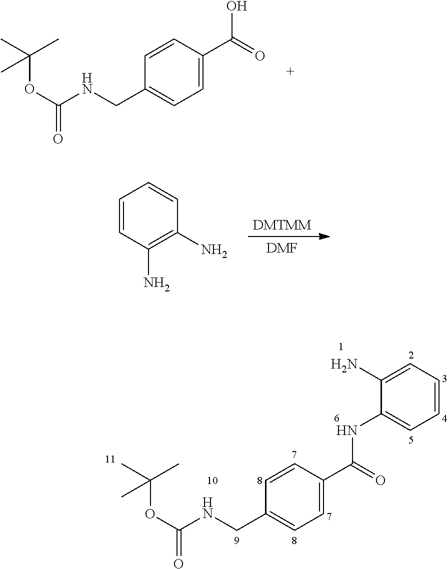

To a stirred solution of 4-{[(tert-butoxycarbonyl)amino]methyl}benzoic acid (2.37 g, 9.44 mmol) and 4-(4,6-dimethoxy-1,3,5-triazin-2-yl)-4-methylmorpholin-4-ium chloride (2.60 g, 9.44 mmol) in DMF (30 mL) was added o-phenylenediamine (1.43 g, 13.22 mmol). After stirring for 20 h at room temperature, the mixture was diluted with water (150 mL) and extracted with EtOAc (2×40 mL). The combined extracts were washed with water (40 mL), dried (MgSO₄), filtered and concentrated in vacuo. The oily residue was triturated with Et₂O to give the product as pale yellow needles (2.25 g, 70%), m.p. 160-162° C.

δH (DMSO-d₆, 500 MHz): 9.63 (s, 1H, H-6), 7.95 (d, J 7.7, 2H, H-7), 7.50 (s, b, 1H, H-10), 7.37 (d, J 7.7, 2H, H-8), 7.18 (d, J 7.4, 1H, H-5), 6.98 (dd, J 7.8, 7.3, 1H, H-3), 6.80 (d, J 7.8, 1H, H-2), 6.61 (dd, J 7.4, 7.3, 1H, H-4), 4.90 (s, b, 2H, H-1), 4.21 (d, J 5.7, 2H, H-9), 1.42 (s, 9H, H-11).

δC (DMSO-d₆, 75 MHz): 165.5, 156.2, 144.1, 143.5, 133.4, 128.1, 127.0, 126.8, 123.7, 116.6, 116.5, 78.3, 43.5, 28.6.

v/max (film) 3419, 3365, 3325, 2989, 1686, 1651, 1655, 1526.

m/z (EZ) (%) 342 (MH⁺, 100).

### Intermediate 4: Ethyl 3-(2-iodophenyl)acrylate

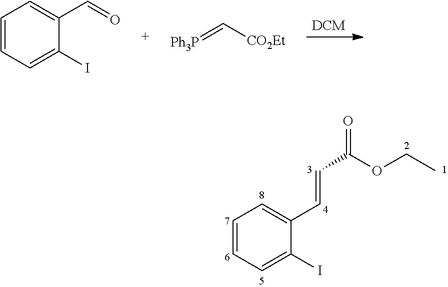

2-Iodobenzaldehyde (2.11 g, 9.1 mmol) and ethyl (triphosphoranylidene)acetate (3.56 g, 10.2 mmol) were combined in DCM (50 mL) and stirred at room temperature for 1 h. The mixture was concentrated in vacuo and the residue filtered through a silica pad eluting with 9:1 v/v Et₂O:pet. ether to afford the product as a yellow oil (2.53 g, 92%, 5.8:1 E:Z).

δH (CDCl₃, 500 MHz): E isomer: 7.91 (d, J 15.8, 1H, H-4), 7.91 (d, J 7.9, 1H, H-8), 7.56 (d, J 7.7, 1H, H-5), 7.36 (dd, J 7.7, 7.5, 1H, H-6), 7.04 (dd, J 7.9, 7.5, 1H, H-7), 6.31 (d, J 15.8, 1H, H-3), 4.28 (q, J 7.1, 2H, H-2), 1.35 (t, J 7.1, 3H, H-1).

Z isomer: 7.85 (d, J 7.9, 1H, H-8), 7.41 (d, J 7.8, 1H, H-5), 7.31 (dd, J 7.8, 7.5, 1H, H-6), 7.01 (dd, 7.9, 7.5, 1H, H-7), 6.97 (d, J 12.1, 1H, H-4), 6.02 (d, J 12.1, 1H, H-3), 4.09 (q, J 7.1, 2H, H-2), 1.15 (t, J 7.1, 3H, H-1).

δC (CDCl₃, 75 MHz): 166.7, 166.1, 148.1, 146.9, 140.4, 140.0, 138.9, 138.3, 131.6, 130.5, 130.2, 129.0, 127.9, 127.8, 121.8, 121.7, 101.6, 61.1, 60.7, 14.7, 14.4.

v/max (film) 2979, 1712, 1635, 1461.

### Intermediate 5: 3-(2-Iodophenyl)-1-phenylprop-2-en-1-one

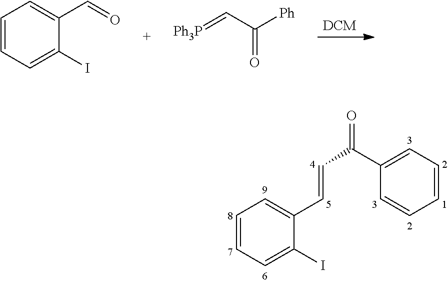

2-Iodobenzaldehyde (1.16 g, 5 mmol) and (triphenylphosphoranylidene)acetophenone (2.28 g, 6 mmol) were combined in DCM and stirred at reflux for 24 h, and the solvent removed in vacuo. Column chromatography (30 g SiO₂, 8:1 v/v hexane:Et₂O) afforded the product as a yellow oil (1.51 g, 90%, 3:1 E:Z).

δH (CDCl₃, 500 MHz): E isomer: 8.03 (d, J 7.3, 2H, H-3), 7.98 (d, J 15.8, 1H, H-5), 7.93 (d, J 7.7, 1H, H-9), 7.69 (d, J 7.7, 1H, H-6), 7.60 (t, J 7.5, 1H, H-1), 7.51 (dd, J 7.5, 7.3, 2H, H-2), 7.40 (t, J 7.7, 1H, H-7), 7.34 (d, J 15.8, 1H, H-4), 7.08 (t, J 7.7, 1H, H-8). Z isomer: 7.86 (d, J 7.3, 2H, H-3), 7.77 (d, J 8.1, 1H, H-9), 7.46 (t, J 7.3, 1H, Ar—H), 7.35 (m, 2H, 2×Ar—H), 7.23 (d, J 7.7, 1H, H-6), 7.09 (m, 2H, Ar—H, H-5), 6.86 (dd, J 8.5, 7.3, 1H, Ar—H), 6.67 (d, J 12.4, 1H, H-4).

δC (CDCl₃, 75 MHz): 194.1, 190.4, 147.9, 143.1, 140.1, 139.5, 138.7, 138.4, 137.8, 137.1, 133.1, 132.9, 131.3, 130.4, 129.7, 128.9, 128.7, 128.6, 128.4, 128.3, 127.8, 127.7, 127.5, 125.3, 101.7, 98.7.

v/max (film) 3058, 1663, 1604, 1578, 1459.

### Intermediate 6: 1-[(2E)-3-(2-Iodophenyl)prop-2-enoyl]piperidine

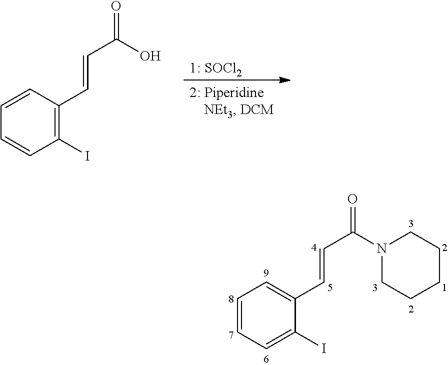 [SMILES: O=C(O)/C=C/c1ccccc1I ; O=C(/C=C/c1ccccc1I)N1CCCCC1]

2-Iodocinnamic acid (1.37 g, 5 mmol) and SOCl₂ (6 mL) were stirred together at reflux for 2 h, and the excess SOCl₂ removed in vacuo. DCM (2×10 mL) was used to azeotrope residual traces of SOCl₂. The solid residue was dissolved in DCM (10 mL), and piperidine (0.85 g, 10 mmol) and NEt₃ (2.02 g, 20 mmol) added successively, dropwise over 1 min each at 0° C. with stirring. The resulting mixture was stirred for 18 h, washed successively with HCl (1M aq., 10 mL), NaHCO₃ (sat. aq., 10 mL) and brine (10 mL), dried (MgSO₄), filtered and concentrated in vacuo. The residue was triturated with hexane to give the product as an off-white solid (1.17 g, 66%).

δH (CDCl₃, 500 MHz): 7.88 (d, J 7.7, 1H, H-9), 7.74 (d, J 15.2, 1H, H-5), 7.52 (dd, J 7.7, 1.3, 1H, H-6), 7.34 (dd, J 7.7, 7.3, 1H, H-7), 7.01 (ddd, J 7.7, 7.3, 1.3, 1H, H-8), 6.72 (d, J 15.2, 1H, H-4), 3.66 (s, b, 2H, H-3), 3.58 (s, b, 2H, H-3), 1.72-1.60 (m, 6H, H-1, H-2).

δC (CDCl₃, 75 MHz): 164.9, 144.9, 139.9, 139.2, 130.4, 128.4, 127.2, 121.5, 100.6, 47.2, 26.8, 24.6.

v/max (film) 2940, 2857, 1643, 1601, 1464, 1442.

### Intermediate 7: 4-(Aminomethyl)-N-(2-aminophenyl)benzamide

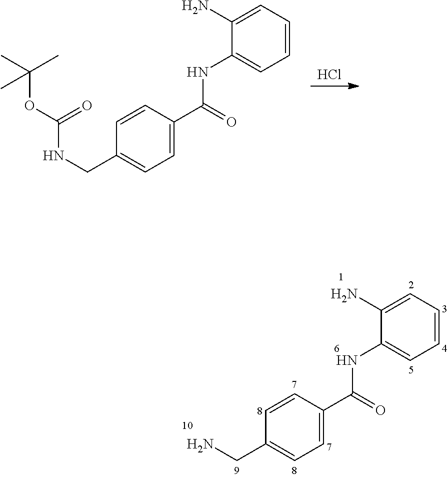 [SMILES: CC(C)(C)OC(=O)NCc1ccc(C(=O)Nc2ccccc2N)cc1 ; Cl ; NCc1ccc(C(=O)Nc2ccccc2N)cc1]

Tert-Butyl (4-{[(2-aminophenyl)amino]carbanyl}benzyl)carbamate (2.0 g, 5.87 mmol) was dissolved in HCl (37% aq., 20 mL) with stirring at room temperature. After stirring at room temperature for 1.5 h, the mixture was cooled to 0° C. and basified with KOH pellets, added singly over 20 min, to pH 14. The resulting yellow precipitate was collected by filtration, dissolved in hot THF (50 mL), and filtered. The solid was re-dissolved in hot THF (50 mL) and filtered. The combined filtrates were concentrated in vacuo to give the product as a pale yellow amorphous solid (1.23 g, 87%).

Found: 242.1286; C₁₄H₁₆N₃O requires 242.1288.

δH (CD₃OD, 500 MHz): 7.97 (d, J 8.1, 2H, H-7), 7.51 (d, J 8.1, 2H, H-8), 7.10 (d, J 7.7, 1H, H-5), 7.00 (dd, J 7.3, 7.2, 1H, H-3), 6.83 (d, J 7.2, 1H, H-2), 6.69 (dd, J 7.7, 7.3, 1H, H-4), 4.11 (s, 2H, H-9).

δC (CD₃OD, 75 MHz): 168.3, 143.8, 138.4, 136.6, 130.4, 130.0, 129.0, 128.0, 125.6, 120.2, 119.2, 44.2.

v/max (film) 3260, 1636, 1526.

m/z (EZ) (%) 242 (100, MH⁺), 505 (2MNa⁺)

### Intermediate 8: (2E)-N-(2-Aminophenyl)-3-(2-iodophenyl)acrylamide

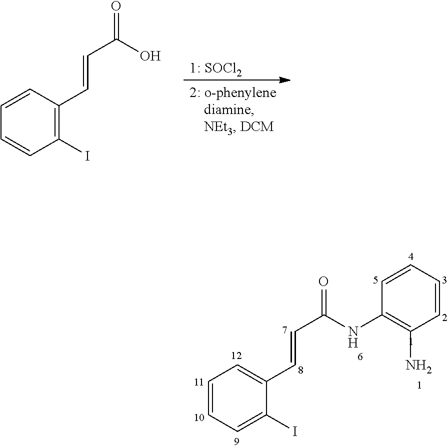 [SMILES: O=C(O)/C=C/c1ccccc1I ; Nc1ccccc1NC(=O)/C=C/c1ccccc1I]

2-Iodocinnamic acid (0.685 g, 2.5 mmol) and SOCl₂ (3 mL) were stirred at reflux for 2 h, and the excess SOCl₂ removed in vacuo. DCM (2×10 mL) was used to azeotrope residual traces of SOCl₂, and the solid residue was dissolved in DCM (10 mL). o-Phenylenediamine (0.810 g, 7.5 mmol) was added portionwise over 1 min at 0° C. with stirring, and NEt₃ (1.01 g, 10 mmol) was immediately added dropwise over 1 min at 0° C. After stirring at room temperature for 24 h, the mixture was washed with water (10 mL), NaHCO₃ (sat. aq., 10 mL) and brine (10 mL), dried (MgSO₄), filtered and concentrated in vacuo. Column chromatography of the residue (30 g silica, 2:1 v/v hexanes:EtOAc) gave the product as yellow needles (0.400 g, 44%), m.p. 194-196° C.

(Found: C, 49.5; H, 3.60; N, 7.95; I, 35.15. C₁₅H₁₃N₂I requires: C, 49.47, H, 3.60, N, 7.69, I, 34.85%).

δH (DMSO-d₆, 500 MHz): 9.47 (s, b, 1H, H-6), 7.99 (d, J 7.7, 1H, H-12), 7.71 (d, J 15.4, 1H, H-8), 7.70 (d, J 8.1, 1H, H-9), 7.51 (dd, J 7.7, 7.3, 1H, H-11), 7.38 (d, J 7.5, 1H, H-5), 7.17 (dd, J 8.1, 7.3, 1H, H-10), 6.94 (dd, J 7.7, 7.3, 1H, H-3), 6.84 (d, J 15.4, 1H, H-7), 6.77 (d, J 7.7, 1H, H-2), 6.60 (dd, J 7.5, 7.3, 1H, H-4), 4.99 (s, b, 2H, H-1).

δC (DMSO-d₆, 75 MHz): 163.2, 142.8, 142.0, 140.1, 138.0, 131.5, 129.3, 127.4, 126.3, 125.8, 125.1, 123.6, 116.6, 116.3, 102.0.

v/max (film) 3233, 1648, 1452.

m/z (EZ) (%) 365 (MH⁺, 100).

### Intermediate 9: (2E)-N-(2-Hydroxyethyl)-3-(2-iodophenyl)acrylamide

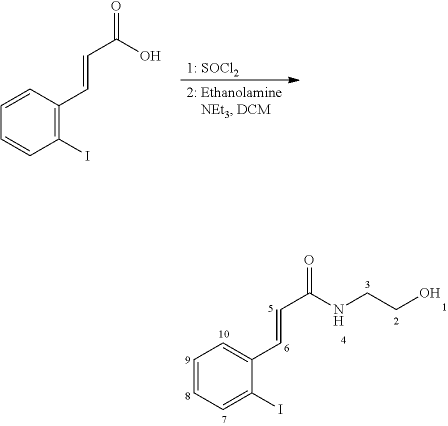 [SMILES: O=C(O)/C=C/c1ccccc1I ; O=C(/C=C/c1ccccc1I)NCCO]

2-Iodocinnamic acid (0.685 g, 2.5 mmol) and SOCl₂ (3 mL) were stirred at reflux for 2 h, and the excess SOCl₂ removed in vacuo. DCM (2×10 mL) was used to azeotrope residual traces of SOCl₂. The solid residue was dissolved in DCM (10 mL), and ethanolamine (0.31 g, 5 mmol) and NEt₃ (1.01 g, 10 mmol) added successively, dropwise over 1 min each at 0° C. with stirring. The resulting mixture was stirred for 18 h, washed successively with HCl (1M aq., 10 mL), NaHCO₃ (sat. aq., 10 mL) and brine (10 mL), dried (MgSO₄), filtered and concentrated in vacuo. The residue was triturated with hexane to give the product as colourless needles (0.44 g, 58%), m.p. 130-132° C.

(Found: C, 41.75; H, 3.80; N, 4.65; I, 40.10. C₁H₁₂INO₂ requires: C, 41.66, H, 3.81, N, 4.42, I, 40.02).

δH (CDCl₃, 500 MHz): 8.26 (s, b, 1H, H-4), 7.94 (d, J 7.5, 1H, H-10), 7.61 (d, J 7.4, 1H, H-7), 7.56 (d, J 15.6, 1H, H-6), 7.46 (dd, J 7.5, 7.3, 1H, H-9), 7.14 (dd, J 7.4, 7.3, 1H, H-8), 6.60 (d, J 15.0, 1H, H-5), 4.78 (s, b, 1H, H-1), 3.48 (s, b, 2H, H-2), 3.27 (t, J 5.5, 2H, H-3).

δC (CDCl₃, 75 MHz): 164.8, 141.8, 140.0, 138.1, 131.3, 129.2, 127.3, 125.7, 101.9, 79.5, 60.1.

v/max (film) 3422, 3281, 1647, 1615.

m/z (EZ) (%) 318 (MH⁺, 100), 145 (100).

### Intermediate 10: Tert-butyl (2-{[(2E)-3-(2-iodophenyl)prop-2-enoyl]amino}ethyl)carbamate

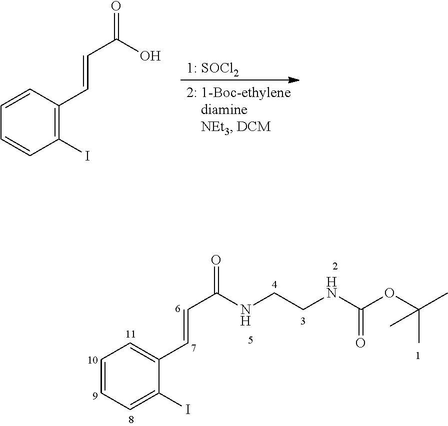 [SMILES: O=C(O)/C=C/c1ccccc1I ; CC(C)(C)OC(=O)NCCNC(=O)/C=C/c1ccccc1I]

2-Iodocinnamic acid (1.37 g, 5 mmol) and SOCl₂ (6 mL) were stirred at reflux for 2 h, and the excess SOCl₂ removed in vacuo. DCM (2×10 mL) was used to azeotrope residual traces of SOCl₂. The solid residue was dissolved in DCM (10 mL), and tert-butyl (2-aminoethyl)carbamate (1.60 g, 10 mmol) and NEt₃ (2.02 g, 20 mmol) added successively, dropwise over 1 min each at 0° C. with stirring. The resulting mixture was stirred for 18 h, washed successively with HCl (1M aq., 10 mL), NaHCO₃ (sat. aq., 10 mL) and brine (10 mL), dried (MgSO₄), filtered and concentrated in vacuo. Crystallisation from DCM/hexane gave the product as pale yellow needles (1.33 g, 66%).

Found: 439.0473; C₁₆H₂₁N₂O₃INa requires 439.0489.

δ_{H} (500 MHz, CDCl₃): 7.89 (d, J 7.5, 1H, H-11), 7.79 (d, J 15.6, 1H, H-7), 7.49 (d, J 7.3, 1H, H-8), 7.32 (t, J 7.3, 1H, H-9), 7.01 (dd, J 7.5, 7.3, 1H, H-10), 6.53 (s, b, 1H, N—H), 6.26 (d, J 15.6, 1H, H-6), 4.99 (s, b, 1H, N—H), 3.53-3.50 (m, 2H, H-4), 3.36-3.35 (m, 2H, H-3), 1.44 (s, 9H, H-1).

δ_{C} (75 MHz, CDCl₃): 166.2, 157.5, 144.5, 140.4, 138.8, 131.1, 128.8, 127.6, 124.3, 101.3, 80.3, 41.7, 40.6, 28.8.

v/max (film) 3343, 3310, 1678, 1647, 1615, 1529.

m/z (%) 439 (MNa⁺, 100).

### Intermediate 11: (2E)-3-(3-Hydroxy-4-methoxyphenyl)-N-(2-{[(2E)-3-(2-iodophenyl)prop-2-enoyl]amino}ethyl)-2-(3,4,5-trimethoxyphenyl)acrylamide

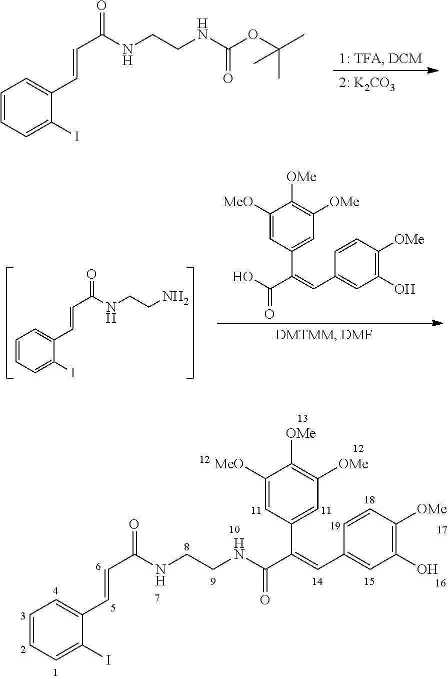

TFA (1 mL) was added dropwise over 1 min to a stirred solution of tert-butyl (2-{[(2E)-3-(2-iodophenyl)prop-2-enoyl]amino}ethyl)carbamate (0.582 g, 1.5 mmol) in DCM (5 mL) at room temperature. After stirring for 1 h, the mixture was concentrated in vacuo, and the residue dissolved in DMF (5 mL). K₂CO₃ (0.276 g, 2 mmol) was added portionwise over 1 min with stirring at room temperature, and the resulting mixture was added in one portion to a solution of (2E)-3-(3-hydroxy-4-methoxyphenyl)-2-(3,4,5-trimethoxy phenyl)acrylic acid (0.540 g, 1.5 mmol) and DMTMM (0.414 g, 1.5 mmol) in DMF (15 mL). The resulting mixture was stirred at room temperature for 18 h, diluted with water (50 mL) and extracted with EtOAc (3×20 mL). The combined organic phases were washed with water (20 mL), dried (MgSO₄), filtered and concentrated in vacuo. Column chromatography (30 g silica, EtOAc) afforded the product as a colourless amorphous solid (0.512 g, 52%).

Found: 659.1250; C₃₀H₃₂N₂O₇I requires 659.1249.

δH (CDCl₃, 500 MHz): 7.86 (d, J 7.8, 1H, H-4), 7.76 (d, J 15.5, 1H, H-5), 7.72 (s, 1H, H-14), 7.50 (d, J 7.6, 1H, H-1), 7.31 (dd, J 7.6, 7.4, 1H, H-2), 7.01 (dd, J 7.8, 7.3, 1H, H-3), 6.90 (s, b, 1H, N—H), 6.65-6.63 (m, 2H, H-15, H-18), 6.57 (dd, J 8.4, 1.6, 1H, H-19), 6.45 (s, 2H, H-11), 6.30 (d, J 15.5, 1H, H-6), 6.04 (s, b, 1H, N—H), 5.65 (s, 1H, H-16), 3.94 (s, 3H, H-17), 3.84 (s, 3H, H-13), 3.82 (s, 6H, H-12), 3.78-3.51 (m, 4H, H-8, H-9).

δ_{C} (75 MHz, CDCl₃): 164.1, 161.1, 149.5, 142.8, 140.4, 139.0, 135.0, 133.4, 133.3, 132.5, 126.9, 126.2, 125.7, 123.5, 123.0, 122.3, 119.2, 118.4, 112.1, 105.6, 101.8, 96.1, 56.2, 51.5, 51.0, 35.9, 35.0.

v/max (film): 3314, 2938, 1660, 1581, 1512.

m/z (EZ) (%) 681 (MNa⁺, 85), 659 (MH⁺, 90), 248 (100).

### Intermediate 12: 4-{[1-(Cyanomethyl)-4-methylene-3,4-dihydroisoquinolin-2(1H)-yl]sulfonyl}benzoic acid

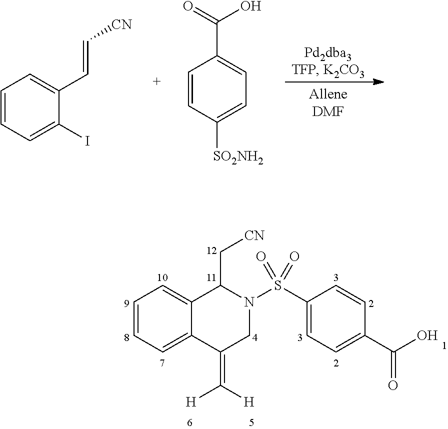

Prepared by the general procedure from 2-iodocinnamonitrile (0.255 g, 1.0 mmol), 4-(aminosulfanyl)benzoic acid (0.201 g, 1.0 mmol), Pd₂dba₃ (0.022 g, 0.025 mmol), TFP (0.023 g, 0.1 mmol), K₂CO₃ (0.276 g, 2.0 mmol) and allene (0.5 bar) in DMF (10 mL), heated at 80° C. for 24 h. Column chromatography (40 g silica, 2:1 v/v Et₂O:hexanes) afforded the product as an amorphous pale yellow solid (0.195 g, 53%).

Found: 391.0717; C₁₉H₁₆N₂O₄SNa requires 391.0723.

δH (DMSO-d₆, 500 MHz): 7.79 (d, J 8.4, 2H, H-3), 7.73 (d, J 8.4, 2H, H-2), 7.43 (d, J 7.8, 1H, H-7), 7.34 (d, J 7.7, 1H, H-10), 7.22 (dd, J 7.5, 7.4, 1H, H-9), 7.12 (dd, J 7.8, 7.4, 1H, H-8), 5.51 (s, 1H, H-6), 5.48 (dd, J 10.1, 4.4, 1H, H-11), 5.13 (s, 1H, H-5), 4.53 (d, J 16.9, 1H, H-4), 4.41 (d, J 16.9, 1H, H-4), 3.34 (dd, J 17.1, 10.1, 1H, H-12), 3.08 (dd, J 17.1, 4.4, 1H, H-12).

δC (DMSO-d₆, 75 MHz): 166.3, 142.5, 134.6, 134.0, 132.2, 130.8, 129.7, 128.6, 128.1, 127.9, 127.7, 124.0, 118.3, 111.3, 53.5, 45.0, 24.7.

v/max (film): 3386, 1697, 1342, 1160.

m/z (%) 391 (MNa⁺, 100).

### Intermediate 13: Tert-butyl {2-[(4-{[1-(cyanomethyl)-4-methylene-3,4-dihydroisoquino-2(1H)-yl]sulfonyl}benzoyl)amino]phenyl}carbamate

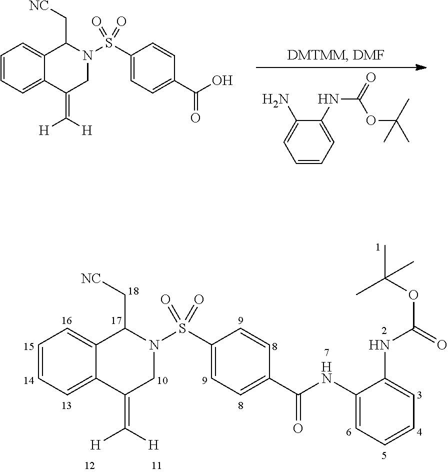 [SMILES: C=C1CN(S(=O)(=O)c2ccc(C(=O)O)cc2)C(CC#N)c2ccccc21 ; CC(C)(C)OC(=O)Nc1ccccc1N ; C=C1CN(S(=O)(=O)c2ccc(C(=O)Nc3ccccc3NC(=O)OC(C)(C)C)cc2)C(CC#N)c2ccccc21]

To a stirred suspension of 4-{[1-(cyanomethyl)-4-methylene-3,4-dihydroisoquinolin-2(1H)-yl]sulfonyl}benzoic acid (0.368 g, 1.0 mmol) and DMTMM (0.276 g, 1.0 mmol) in DMF (5 mL) was added tert-butyl (2-aminophenyl)carbamate (0.208 g, IA mmol) portionwise over 1 min. The resulting mixture was stirred at room temperature for 16 hours, diluted with H₂O (40 mL) and extracted with EtOAc (3×10 mL). The combined organic extracts were washed with water (10 mL), dried (MgSO₄), filtered and concentrated in vacuo. Column chromatography (30 g SiO₂, 2:1 v/v Et₂O/petroleum ether) gave the product as colourless plates (0.340 g, 61%), m.p. 186-188° C.

(Found: C, 64.25; H, 5.45; N, 9.95; S, 5.45. C₃₀H₃₃N₄O₅S requires: C, 64.50, H, 5.41, N, 10.03, S, 5.74).

δH (CDCl₃, 500 MHz): 9.50 (s, b, 1H, H-2), 7.85 (d, J 8.1, 2H, H-9), 7.84 (s, b, 1H, H-7), 7.73 (d, J 8.1, 2H, H-8), 7.43 (d, J 8.1, 1H, H-16), 7.30-7.11 (m, 6H, 6×Ar—H), 6.67-6.66 (m, 1H, Ar—H), 5.51 (s, 1H, H-12), 5.32 (dd, J 6.8, 6.4, 1H, H-17), 5.10 (s, 1H, H-11), 4.54 (d, J 16.7, 1H, H-10), 4.31 (d, J 16.7, 1H, H-10), 3.00 (dd, J 16.7, 6.4, 1H, H-18), 2.92 (dd, J 16.7, 6.8, 1H, H-18), 1.50 (s, 9H, H-1).

δC (CDCl₃, 75 MHz): 164.0, 155.3, 142.0, 138.7, 134.8, 131.7, 131.6, 131.0, 129.9, 129.3, 129.2, 128.2, 127.9, 127.2, 126.7, 126.6, 126.2, 125.1, 116.9, 112.0, 82.3, 54.0, 46.2, 28.7, 26.4.

v/max (film) 2979, 1656, 1524.

m/z (EZ) (%) 581 (MNa⁺, 100).

### Example 1

### Ethyl [2-(4-{[(2-aminophenyl)amino]carbonyl}benzyl)-4-methylene-1,2,3,4-tetrahydroisoquinolin-1-yl]acetate

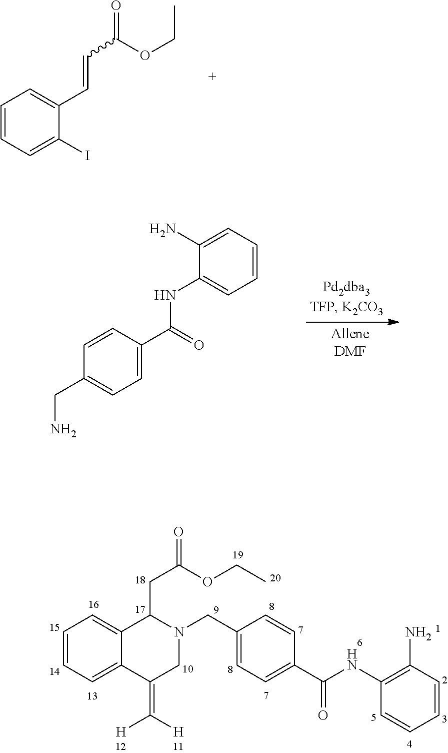 [SMILES: CCOC(=O)C=Cc1ccccc1I ; NCc1ccc(C(=O)Nc2ccccc2N)cc1 ; C=C1CN(Cc2ccc(C(=O)Nc3ccccc3N)cc2)C(CC(=O)OCC)c2ccccc21]

Prepared by the general procedure from ethyl 3-(2-iodophenyl)acrylate (0.302 g, 1.0 mmol), 4-(aminomethyl)-N-(2-aminophenyl)benzamide (0.265 g, 1.1 mmol), Pd₂dba₃ (0.022 g, 0.025 mmol), TFP (0.023 g, 0.1 mmol), K₂CO₃ (0.276 g, 2.0 mmol) and allene (0.5 bar) in DMF (10 mL), heated at 80° C. for 24 h. Column chromatography (30 g silica, 3:2 v/v hexanes:EtOAc) afforded the product as a pale yellow solid. Crystallisation from DCM/hexane gave the product as pale yellow needles (0.241 g, 53%),

m.p. 111-113° C.

(Found: C, 72.80; H, 6.55; N, 9.15. C₂₈H₂₉N₃O₃.⅓H₂O requires: C, 72.86, H, 6.48, N, 9.10).

Found: 456.2271; C₂₈H₃₀N₃O₃ (MH⁺) requires 456.2282.

δH (CDCl₃, 500 MHz): 7.84 (d, J 7.9, 2H, H-7), 7.83 (s, 1H, H-6), 7.70 (d, J 7.3, 1H, H-16), 7.40 (d, J 7.9, 2H, H-8), 7.33 (d, J 7.7, 1H, Ar—H), 7.27-7.25 (m, 2H, 2×Ar—H), 7.12-7.08 (m, 2H, 2×Ar—H), 6.87-6.83 (m, 2H, 2×Ar—H), 5.70 (s, 1H, H-12), 4.94 (s, 1H, H-11), 4.31 (dd, J 10.3, 5.0, 1H, H-17), 4.29-4.11 (m, 2H, H-19), 3.92 (d, J 15.8, 1H, H-10), 3.89 (s, b, 2H, H-1), 3.75 (d, J 13.7, 1H, H-9), 3.66 (d, J 13.7, 1H, H-9), 3.24 (d, J 15.8, 1H, H-10), 2.89 (dd, J 14.6, 10.3, 1H, H-18), 2.65 (dd, J 14.6, 5.1, 1H, H-18), 1.27 (t, J 7.1, 3H, H-20).

δC (CDCl₃, 75 MHz): 171.5, 165.7, 143.6, 140.7, 136.4, 135.0, 132.9, 132.2, 129.2, 128.4, 127.7, 127.1, 125.1, 124.6, 123.7, 119.8, 118.4, 11.0, 60.6, 60.0, 57.5, 49.4, 41.9, 14.3.

v/max (film): 3420, 3344, 3069, 2983, 1724, 1655.

m/z (EZ) (%): 456 (MH⁺, 100).

### Example 2

### N-(2-Aminophenyl)-4-{[4-methylene-1-(2-oxo-2-piperidin-1-ylethyl)-3,4-dihydroisoquinolin-2(1H)-yl]methyl}benzamide

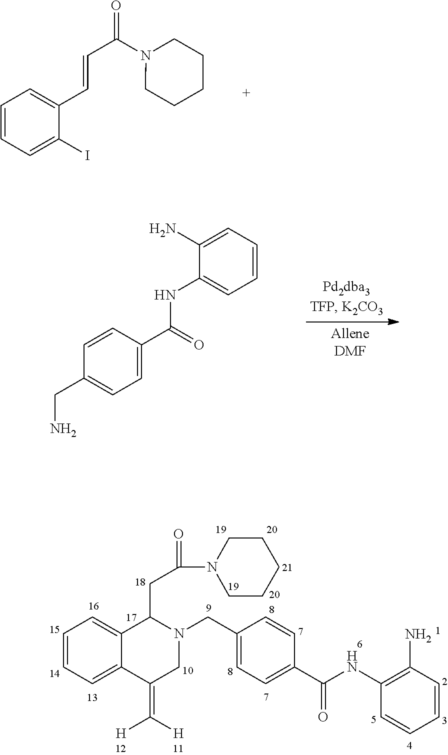 [SMILES: O=C(/C=C/c1ccccc1I)N1CCCCC1 ; NCc1ccc(C(=O)Nc2ccccc2N)cc1 ; C=C1CN(Cc2ccc(C(=O)Nc3ccccc3N)cc2)C(CC(=O)N2CCCCC2)c2ccccc21]

Prepared by the general procedure from 1-[(2E)-3-(2-iodophenyl)prop-2-enoyl]piperidine (0.341 g, 1.0 mmol), 4-(aminomethyl)-N-(2-aminophenyl)benzamide (0.265 g, 1.1 mmol), Pd₂dba₃ (0.022 g, 0.025 mmol), TFP (0.023 g, 0.1 mmol), K₂CO₃ (0.276 g, 2.0 mmol) and allene (0.5 bar) in DMF (10 mL), heated at 80° C. for 24 h. Column chromatography (30 g silica, 1:3 v/v hexanes:EtOAc) afforded the product as a pale yellow foam. Crystallisation from DCM/hexane gave the product as pale yellow plates (0.310 g, 63%), m.p. 89-91° C.

Found: 495.2751; C₃₁H₃₅N₄O₂ (MH⁺) requires 495.2755.

δH (CDCl₃, 500 MHz): 7.84 (d, J 7.8, 2H, H-7), 7.84 (s, b, 1H, H-6), 7.68 (d. J 6.8, 1H, H-16), 7.43 (d, J 7.8, 2H, H-8), 7.34 (d, J 7.1, 1H, Ar—H), 7.28-7.25 (m, 2H, 2×Ar—H), 7.17 (t, J 6.8, 1H, H-15), 7.10 (dd, J 7.3, 6.8, 1H, Ar—H), 6.87-6.84 (m, 2H, 2×Ar—H), 5.70 (s, 1H, H-12), 4.94 (s, 1H, H-11), 4.43 (dd, J 7.7, 6.1, 1H, H-17), 3.94-3.89 (m, 1H, H-19), 3.87 (d, J 15.4, 1H, H-10), 3.77 (d, J 13.4, 1H, H-9), 3.70 (d, J 13.4, 1H, H-9), 3.54-3.50 (m, 1H, H-19), 3.38-3.34 (m, 1H, H-19), 3.28 (d, J 15.4, 1H, H-10), 3.22-3.17 (m, 1H, H-19), 2.99 (dd, J 14.5, 7.7, 1H, H-18), 2.64 (dd, J 14.5, 6.1, 1H, H-18), 1.65-1.55 (m, 4H, H-20), 0.97-0.84 (m, 2H, H-21).

(CDCl₃, 75 MHz): 169.3, 143.7, 140.7, 136.7, 136.1, 132.0, 129.4, 128.3, 128.1, 127.2, 126.9, 124.6, 123.5, 119.8, 118.4, 109.8, 60.2, 57.7, 49.9, 47.1, 43.0, 39.2, 26.4, 25.6, 24.5.

v/max (film): 3265, 2938, 2856, 1618, 1503, 1450.

m/z (EZ) (%): 495 (MH⁺, 100).

### Example 3

### Methyl [2-(4-{[(2-aminophenyl)amino]carbonyl}benzyl)-4-methylene-1,2,3,4-tetrahydroisoquinolin-1-yl]acetate

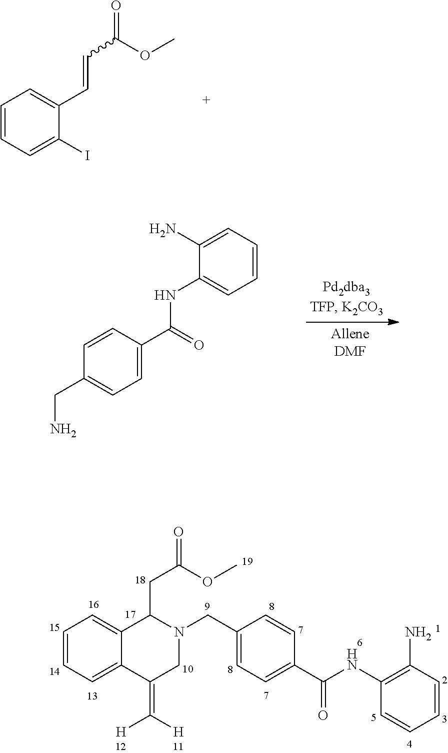 [SMILES: COC(=O)C=Cc1ccccc1I ; NCc1ccc(C(=O)Nc2ccccc2N)cc1 ; C=C1CN(Cc2ccc(C(=O)Nc3ccccc3N)cc2)C(CC(=O)OC)c2ccccc21]

Prepared by the general procedure from methyl 3-(2-iodophenyl)acrylate (0.288 g, 1.0 mmol), 4-(aminomethyl)-N-(2-aminophenyl)benzamide (0.265 g, 1.1 mmol), Pd₂dba₃ (0.022 g, 0.025 mmol), TFP (0.023 g, 0.1 mmol), K₂CO₃ (0.276 g, 2.0 mmol) and allene (0.5 bar) in DMF (10 mL), heated at 80° C. for 24 h. Column chromatography (30 g silica, 3:2 v/v hexanes:EtOAc) afforded the product as a pale yellow foam. Crystallisation from DCM/hexane gave the product as pale yellow needles (0.302 g, 68%), m.p. 151-153° C.

(Found: C, 72.8; H, 6.10; N, 9.15. C₂₇H₂₇N₃O₃.0.25H₂O requires: C, 72.71, H, 6.21, N, 9.42).

δH (CDCl₃, 500 MHz): 7.85 (d, J 7.9, 2H, H-7), 7.85 (s, b, 1H, H-6), 7.70 (d, J 7.3, 1H, H-10), 7.39 (d, J 7.9, 2H, H-8), 7.34 (d, J 7.6, 1H, Ar—H), 7.27-7.25 (m, 2H, 2×Ar—H), 7.12-7.09 (m, 2H, 2×Ar—H), 6.87-6.85 (m, 2H, 2×Ar—H), 5.70 (s, 1H, H-12), 4.96 (s, 1H, H-11), 4.29 (dd, J 10.4, 5.0, 1H, H-17), 3.93 (d, J 15.7, 1H, H-10), 3.89 (s, b, 2H, H-1), 3.75 (d, J 14.0, 1H, H-9), 3.73 (s, 3H, H-19), 3.65 (d, J 14.0, 1H, H-9), 3.25 (d, J 15.7, 1H, H-10), 2.90 (dd, J 14.6, 10.5, 1H, H-18), 2.66 (dd, J 14.6, 5.0, 1H, H-18).

δC (CDCl₃, 75 MHz): 171.9, 165.4, 143.5, 140.7, 136.3, 134.9, 133.0, 132.2, 129.3, 128.4, 127.7, 127.2, 127.1, 125.1, 124.6, 123.7, 119.8, 118.4, 110.1, 59.7, 57.5, 51.8, 49.5, 41.7.

v/max (film): 3347, 1732, 1637.

m/z (EZ) (%): 442 (MH⁺, 100).

### Example 4

### N-(2-Aminophenyl 4-{[4-methylene-1-(2-morpholin-4-yl-2-oxoethyl)-3,4-dihydroisoquinolin-2(1H)-yl]methyl}benzamide

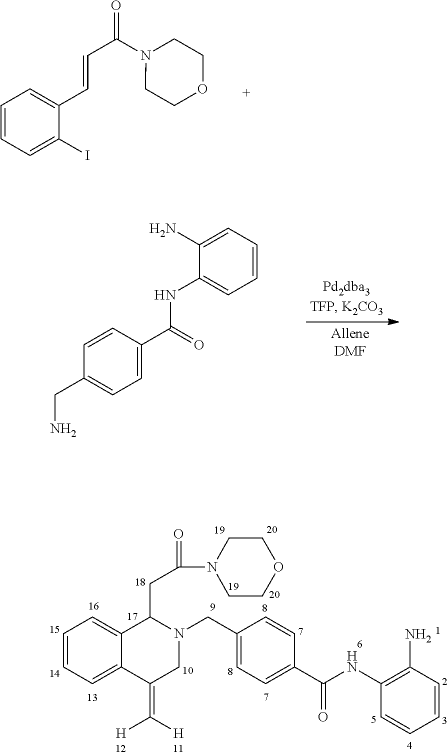 [SMILES: O=C(/C=C/c1ccccc1I)N1CCOCC1 ; NCc1ccc(C(=O)Nc2ccccc2N)cc1 ; C=C1CN(Cc2ccc(C(=O)Nc3ccccc3N)cc2)C(CC(=O)N2CCOCC2)c2ccccc21]

Prepared by the general procedure from 4-[(2E)-3-(2-iodophenyl)prop-2-enoyl]morpholine (0.343 g, 1.0 mmol), 4-(aminomethyl)-N-(2-aminophenyl)benzamide (0.265 g, 1.1 mmol), Pd₂dba₃ (0.022 g, 0.025 mmol), TFP (0.023 g, 0.1 mmol), K₂CO₃ (0.276 g, 2.0 mmol) and allene (0.5 bar) in DMF (10 mL), heated at 80° C. for 24 h. Column chromatography (30 g silica, 1:1 v/v hexanes:EtOAc) afforded the product as a pale yellow foam. Crystallisation from DCM/hexane gave the product as pale yellow needles (0.282 g, 57%), m.p. 99-101° C.

Found: 497.2558; C₃₀H₃₃N₄O₃ (MH⁺) requires 497.2547.

δH (CDCl₃, 500 MHz): 7.88 (s, b, 1H, H-6), 7.85 (d, J 7.8, 2H, H-7), 7.70 (d, J 6.4, 1H, H-16), 7.42 (d, J 7.8, 2H, H-8), 7.35 (d, J 7.8, 1H, Ar—H), 7.28-7.24 (m, 2H, 2×Ar—H), 7.14 (d, J 6.8, 1H, Ar—H), 7.11 (dd, J 7.9, 7.4, 1H, Ar—H), 6.88-6.86 (m, 2H, 2×Ar—H), 5.71 (s, 1H, H-12), 4.97 (s, 1H, H-11), 4.40 (dd, J 6.5, 7.5, 1H, H-17), 3.91 (s, b, 2H, H-1), 3.85 (d, J 15.5, 1H, H-10), 3.77 (d, J 13.4, 1H, H-9), 3.72-3.64 (m, 5H, H-19 & 4×H-20), 3.57-3.52 (m, 1H, H-19), 3.41-3.35 (m, 2H, H-19 & H-9), 3.32 (d, J 15.5, 1H, H-10), 3.19-3.13 (m, 1H, H-19), 2.95 (dd, J 14.2, 7.5, 1H, H-18), 2.67 (dd, J 14.2, 6.5, 1H, H-18).

δC (CDCl₃, 75 MHz): 169.8, 143.4, 140.7, 136.5, 135.7, 133.1, 132.1, 129.4, 128.4, 128.0, 127.3, 127.2, 125.2, 124.6, 123.7, 119.8, 118.4, 110.0, 66.8, 66.4, 60.4, 57.8, 50.1, 16.3, 42.1, 38.8.

v/max (film): 2967, 1623, 1503.

m/z (EZ) (%): 497 (MH⁺, 100).

### Example 5

### N-(2-Aminophenyl)-4-{[4-methylene-1-(2-oxo-2-phenylethyl)-3,4-dihydro isoquinolin-2(1H)-yl]methyl}benzamide

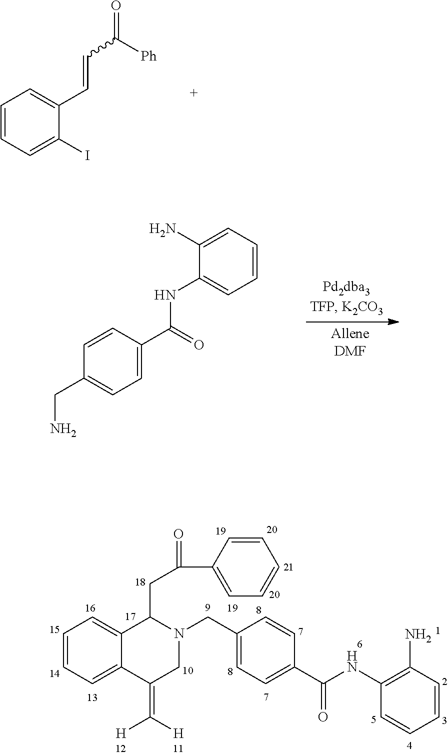 [SMILES: O=C(C=Cc1ccccc1I)c1ccccc1 ; NCc1ccc(C(=O)Nc2ccccc2N)cc1 ; C=C1CN(Cc2ccc(C(=O)Nc3ccccc3N)cc2)C(CC(=O)c2ccccc2)c2ccccc21]

Prepared by the general procedure from 3-(2-iodophenyl)-1-phenylprop-2-en-1-one (0.334 g, 1.0 mmol), 4-(aminomethyl)-N-(2-aminophenyl)benzamide (0.265 g, 1.1 mmol), Pd₂dba₃ (0.022 g, 0.025 mmol), TFP (0.023 g, 0.1 mmol), K₂CO₃ (0.276 g, 2.0 mmol) and allene (0.5 bar) in DMF (10 mL), heated at 80° C. for 24 h. Column chromatography (30 g silica, 2:1 v/v hexanes:EtOAc) afforded the product as an amorphous pale yellow solid (0.240 g, 49%).

Found: 488.2353; C₃₂H₃₀N₃O₂ (MH⁺) requires 488.2333.

δH (CDCl₃, 500 MHz): 7.96 (d, J 7.7, 2H, H-19), 7.78 (s, b, 1H, H-6), 7.74-7.70 (m, 3H, H-13 & H-7), 7.57 (t, J 7.3, 1H, H-21), 7.46 (dd, J 7.7, 7.3, 2H, H-20), 7.32 (d, J 7.7, 1H, Ar—H), 7.28-7.21 (m, 4H, 4×Ar—H), 7.17 (dd, J 3.4, 5.1, 1H, Ar—H), 7.10 (dd, J 7.7, 6.8, 1H, Ar—H), 6.88-6.83 (m, 2H, 2×Ar—H), 5.71 (s, 1H, H-12), 4.95 (s, 1H, H-11), 4.57 (dd, J 8.6, 5.1, 1H, H-17), 3.97 (d, J 15.6, 1H, H-10), 3.87 (s, b, 2H, H-1), 3.71 (d, J 13.7, 1H, H-9), 3.64 (dd, J 15.8, 8.6, 1H, H-18), 3.62 (d, J 13.7, 1H, H-9), 3.23 (d, J 15.6, 1H, H-10), 3.17 (dd, J 15.8, 5.1, 1H, H-18). δC (CDCl₃, 75 MHz): 143.4, 140.7, 137.7, 136.5, 135.9, 133.0, 132.8, 132.2, 129.3, 128.7, 127.8, 127.2, 127.1, 127.0, 125.1, 124.7, 123.7, 119.8, 118.4, 110.0, 59.5, 57.6, 49.8, 45.3.

v/max (film): 3411, 1649.

m/z (EZ) (%): 488 (MH⁺, 100).

### Example 6

### N-(2-Aminophenyl)-4-{[1-(cyanomethyl)-4-methylene-3,4-dihydroisoquinolin-2(1H)-yl]methyl}benzamide

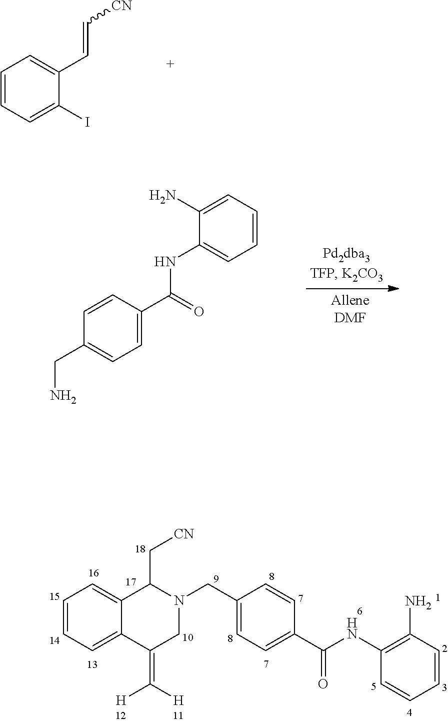 [SMILES: N#CC=Cc1ccccc1I ; NCc1ccc(C(=O)Nc2ccccc2N)cc1 ; C=C1CN(Cc2ccc(C(=O)Nc3ccccc3N)cc2)C(CC#N)c2ccccc21]

Prepared by the general procedure from 2-iodocinnamonitrile (0.255 g, 1.0 mmol), 4-(aminomethyl)-N-(2-aminophenyl)benzamide (0.265 g, 1.1 mmol), Pd₂dba₃ (0.022 g, 0.025 mmol), TFP (0.023 g, 0.1 mmol), K₂CO₃ (0.276 g, 2.0 mmol) and allene (0.5 bar) in DMF (10 mL), heated at 80° C. for 24 h. Column chromatography (30 g silica, 2:1 v/v hexanes:EtOAc) afforded the product as a pale yellow foam. Crystallisation from DCM/hexane gave the product as colourless prisms (0.281 g, 69%), m.p. 93-95° C.

(Found: C, 76.2; H, 5.9; N, 14.0. C₂₆H₂₄N₄O requires: C, 76.45, H, 5.92, N, 13.72).

δH (CDCl₃, 500 MHz): 7.89 (d, J 8.4, 2H, H-7), 7.87 (s, b, 1H, H-6), 7.72 (d, J 7.1, 1H, H-13), 7.54 (d, J 8.4, 2H, H-8), 7.36-7.27 (m, 3H, 3×Ar—H), 7.13-7.06 (m, 2H, 2×Ar—H), 6.88-6.84 (m, 2H, 2×Ar—H), 5.76 (s, 1H, H-12), 5.04 (s, 1H, H-11), 4.07 (dd, J 9.8, 5.2, 1H, H-17), 3.91 (d, J 15.5, 1H, H-10), 3.87 (s, b, 2H, H-1), 3.86 (d, J 14.0, 1H, H-9), 3.71 (d, J 14.0, 1H, H-9), 3.36 (d, J 15.5, 1H, H-10), 2.91 (dd, J 17.0, 9.8, 1H, H-18), 2.69 (dd, J 17.0, 5.2, 1H, H-18).

δC (CDCl₃, 75 MHz): 165.7, 142.6, 140.7, 135.7, 133.4, 132.7, 132.3, 129.3, 128.7, 128.0, 127.8, 127.5, 127.2, 125.2, 124.7, 124.0, 119.8, 118.4, 118.2, 110.8, 58.5, 57.6, 50.0, 24.8.

v/max (film): 3434, 3322, 1639, 1524, 2252.

m/z (EZ) (%): 409 (MH⁺, 35), 225 (fragment C₁-C₉, 100)

### Example 7

### N-(2-Aminophenyl)-4-[(4-methylene-1-oxo-3,4-dihydroisoquino-2(1H)-yl)methyl]benzamide

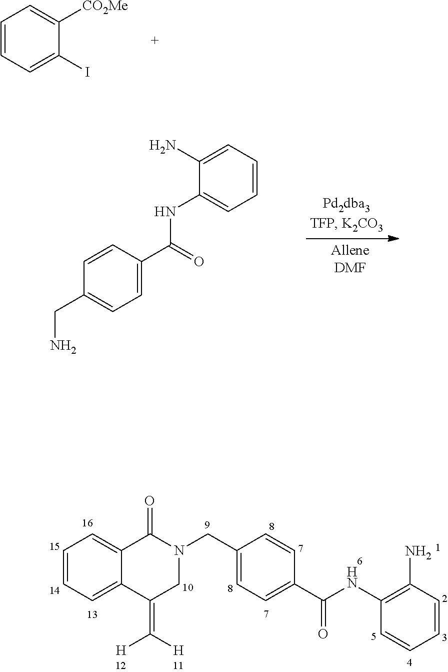 [SMILES: COC(=O)c1ccccc1I ; NCc1ccc(C(=O)Nc2ccccc2N)cc1 ; C=C1CN(Cc2ccc(C(=O)Nc3ccccc3N)cc2)C(=O)c2ccccc21]

Prepared by the general procedure from methyl 2-iodobenzoate (0.131 g, 0.5 mmol), 4-(aminomethyl)-N-(2-aminophenyl)benzamide (0.133 g, 0.55 mmol), Pd₂dba₃ (0.011 g, 0.0125 mmol), TFP (0.012 g, 0.05 mmol), K₂CO₃ (0.138 g, 1.0 mmol) and allene (0.5 bar) in DMF (10 mL), heated at 80° C. for 24 h. Column chromatography (25 g silica, 1:1 v/v hexanes:EtOAc) afforded the product as a pale orange solid. Crystallisation from DCM/hexane gave the product as pale orange plates (0.115 g, 60%), m.p. 202-204° C.

(Found: C, 74.55; H, 5.50; N, 10.60. C₂₄H₂₁N₃O₂.0.25H₂O requires: C, 74.30, H, 5.59, N, 10.83).

δH (CDCl₃, 500 MHz): 8.21 (d, J 7.8, 1H, H-16), 7.88 (d, J 7.8, 2H, H-7), 7.84 (s, b, 1H, H-6), 7.57 (d, J 7.6, 1H, Ar—H), 7.53 (dd, J 7.4, 7.7, 1H, Ar—H), 7.48-7.44 (m, 3H, 3×Ar—H), 7.33 (d, J 7.7, 1H, Ar—H), 7.10 (dd, J 6.8, 7.1, 1H, Ar—H), 6.87-6.84 (m, 2H, 2×Ar—H), 5.59 (s, 1H, H-12), 5.18 (s, 1H, H-11), 4.89 (s, 2H, H-9), 4.16 (s, 2H, H-10), 3.86 (s, b, 2H, H-1).

δC (CDCl₃, 75 MHz): 165.5, 163.7, 141.1, 140.7, 136.3, 135.8, 133.6, 132.3, 129.0, 128.7, 128.2, 127.8, 127.5, 127.2, 125.2, 124.6, 123.2, 119.8, 118.4, 112.6, 52.0, 50.0.

v/max (film): 3400, 1632, 1530, 1501.

m/z (EZ) (%): 384 (100, MH⁺), 406 (82, MNa⁺), 789 (2MNa⁺).

### Example 7

### Alternative synthesis of N-(2-Aminophenyl)-4-[(4-methylene-1-oxo-3,4-dihydroisoquinolin-2(1H)-yl)methyl]benzamide

### General Materials and Methods

LCMS analyses were carried out using the following equipment and method:
Liquid handler and SFO: Alliance e2695.
UV detector: Waters 2998. The detection was done at 254 nm and using an array between 210-600 nm. The diode array trace was used to measure purity.
Mass spectrometer: Acquity SQ, detecting masses in the range of 100 and 700 g/mol.
Column: 5 micron SunFire C18, 50×4.60 mm0
Injection volume: 10 μL.
Flow rate^{.} 1.5 mL/min
Mobile phase A: Water+0.1% Formic acid
Mobile phase B: Acetonitrile+0.1% Formic acid.

| Time/min. | % Mobile Phase A | % Mobile Phase B |
|---|---|---|
| 0.00 | 95 | 5 |
| 10.00 | 5 | 95 |
| 10.50 | 5 | 95 |
| 10.60 | 95 | 5 |
| 12.00 | 95 | 5 |

NMR spectra were acquired on an Oxford Instruments AS400 9.4 Tesla 400 MHz magnet with either a 5 mm BBO or PH SEF 400SB F—H-D-05 probe and an AVANCE/DPX400 console. Deuterated solvents were from Sigma-Aldrich or Acros organics. Chemical shifts are in ppm. Laboratory chemicals were purchased from commercial sources and used without further purification.
Names were generated using Autonom 2000.

### Step 1—Preparation of [4-(2-Amino-phenylcarbamoyl)-benzyl]-carbamic acid tert-butyl ester

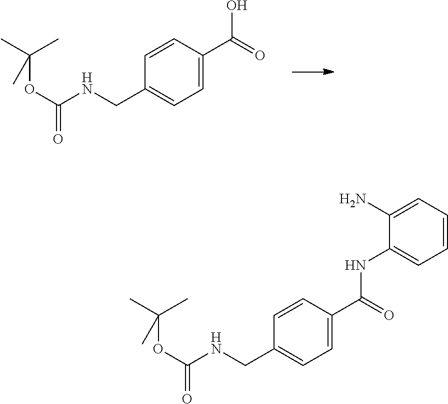 [SMILES: CC(C)(C)OC(=O)NCc1ccc(C(=O)O)cc1 ; CC(C)(C)OC(=O)NCc1ccc(C(=O)Nc2ccccc2N)cc1]

4-(Boc-aminomethyl)benzoic acid (10.09 g, 40.154 mmol) and 4-(4,6-Dimethoxy-1,3,5-triazin2-yl)-4-methylmorpholinium chloride (11.11 g, 40.149 mmol, 1.0 equivalents) were suspended in DMF (130 ml) and stirred at ambient. To this was added 1,2 phenelynediamine (6.08 g, 56.223 mmol, 1.4 equivalents), and the addition rinsed in with 20 ml DMF. The reaction was stirred at ambient overnight, after which time LCMS analysis indicated 95% conversion to product. The reaction mixture was poured into 150 ml 1M NaOH solution and extracted with 3×200 ml ethyl acetate. The ethyl acetate layers were combined and washed with 2×200 ml water, then dried over magnesium sulphate, filtered and concentrated under reduced pressure to give crude 1, 13.88 g (101%). This crude solid was suspended in 70 ml diethyl ether and stirred at ambient temperature for 24 hours. The solid was isolated by filtration, washed through with 2×10 ml diethyl ether and dried in vacuo at ambient to give 11.99 g (87%) 1, 90% by LCMS.

11.08 g of this was suspended in 110 ml ethyl acetate and stirred at ambient temperature for 4 hours. The solid was isolated by filtration, washed through with 2×10 ml ethyl acetate and dried in vacuo at 40-45° C. to give 1, 8.46 g (76% recovery).

LCMS: Purity >99% Retention time 5.3 minutes MI:M+1=342

NMR (d6-DMSO): 9.62 (s, 1H), 7.93 (d, J=8 Hz, 2H), 7.49 (t, J=6 Hz, 1H), 7.36 (d, J=8 Hz, 2H), 7.17 (d, J=7 Hz, 1H), 6.97 (m, 1H), 6.78 (dd, J=1 Hz, 8 Hz, 1H), 6.60 (m, 1H), 4.89 (s, 2H), 4.20 (d, J=6 Hz, 2H), 1.41 (s, 9H).

### Step 2—Preparation of 4-Aminomethyl-N-(2-amino-phenyl)-benzamide

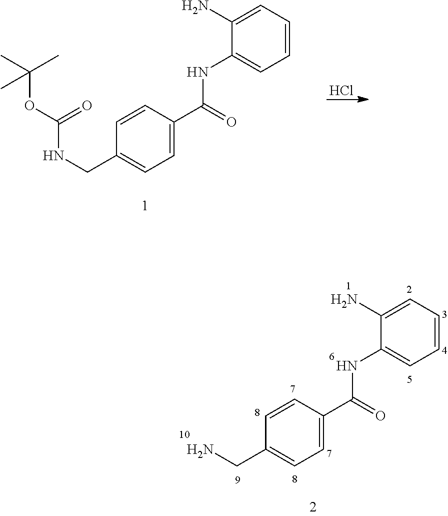 [SMILES: CC(C)(C)OC(=O)NCc1ccc(C(=O)Nc2ccccc2N)cc1 ; Cl ; NCc1ccc(C(=O)Nc2ccccc2N)cc1]

[4-(2-Amino-phenylcarbamoyl)-benzyl]-carbamic acid tert-butyl ester (4.50 g, 13.181 mmol) was suspended in DCM (45 ml) and cooled by ice-bath. To this was added trifluoroacetic acid (23 ml) over a period of 15 minutes, at which time the reaction was removed from cooling and allowed to warm to ambient. The reaction was stirred for a further 2 hours at ambient, LCMS indicating >99% conversion. The reaction mixture was concentrated under reduced pressure and the evaporation residue dissolved in methanol (70 ml) and loaded onto a 70 g Isolute SCX-2 cartridge. This was then eluted with a further three 70 ml portions of methanol. These methanol fractions were discarded to waste. The column was then eluted with 4×70 ml 2M ammonia in methanol, the final three fractions were combined and concentrated under reduced pressure to give 2, 3.24 g (102%).

LCMS: Purity >98% Retention time 0.8 minutes MI:M+1=242

NMR (d4-MeOH, 400 MHz): 7.96 (d, J=8 Hz, 2H), 7.49 (d, J=8 Hz, 2H), 7.18 (dd, J=1 Hz, 8 Hz, 1H), 7.08 (m, 1H), 6.90 (dd, J=1 Hz, 8 Hz, 1H), 6.77 (m, 1H), 3.88 (s, 2H).

This procedure was repeated on 3.97 g (11.628 mmol) of 1, using 40 ml DCM and 18 ml trifluoroacetic acid. This gave 2.85 g (101%), analytical data as above.

### Step 3—Preparation of N-(2-Amino-phenyl)-4-(4-methylene-1-oxo-3,4-dihydro-1H-isoquinolin-2-ylmethyl)-benzamide

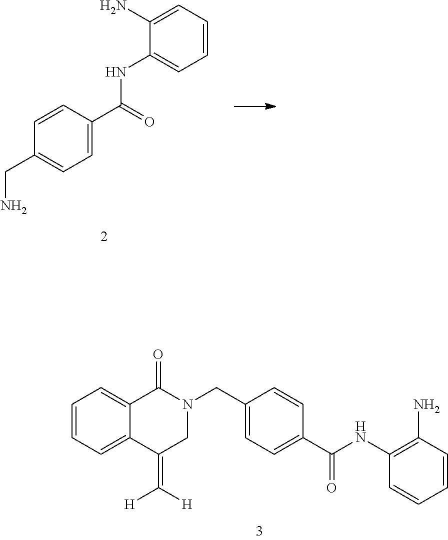 [SMILES: NCc1ccc(C(=O)Nc2ccccc2N)cc1 ; C=C1CN(Cc2ccc(C(=O)Nc3ccccc3N)cc2)C(=O)c2ccccc21]

DMF degassed by sparging with nitrogen for 1 hour. To a Schlenk tube was charged 2 (0.70 g, 2.901 mmol), methyl 2-iodobenzoate (0.68 g, 2.595 mmol, 0.9 equivalents), potassium carbonate (0.72 g, 5.209 mmol, 1.8 equivalents), tri(2-furyl)phosphine (0.067 g, 0.289 mmol, 10 mol %), tris(dibenzylideneacetone)dipalladium(0) (0.066 g, 0.072 mmol, 2.5 mol %) and DMF (70 ml). The Schlenk tube was sealed, then evacuated and backfilled with nitrogen twice. It was then left open to vacuum for 2 minutes. A cylinder of allene was set to 8 psi, the Schlenk tube isolated from vacuum and then opened (whilst still under vacuum) to the allene cylinder, and left open to it for 1 minute. The Schlenk tube was sealed and heated to 80° C. for 21 hours (behind a blast shield). The reaction was left to cool to ambient, then vented. LCMS analysis showed >99% conversion of the methyl 2-iodobenzoate. The Schlenk tube was evacuated and backfilled with nitrogen twice to remove residual allene. The reaction mixture was poured into 3% sodium chloride solution (300 ml) and extracted with ethyl acetate (2×100 ml). The ethyl acetate layers were combined and washed with 3% sodium chloride solution (100 ml), saturated disodium citrate solution (30 ml), 0.1M EDTA disodium salt solution (50 ml) and 3% sodium chloride solution (50 ml). The organics were dried over magnesium sulphate and filtered. To the filtrate was added activated charcoal (0.07 g) and stirred for 35 minutes. The charcoal was removed by filtration through Kieselguhr and the filtrate concentrated under reduced pressure to give crude 3, 1.10 g (99%). This was then columned using a Combiflash Rf (120 g silica column, DCM/MeOH) to give 3, 0.58 g (52%).

This procedure carried out on 4 more batches, the results are summarised in the following table.

| Input/g | Time/hours | Yield/g |
|---|---|---|
| 0.70 | 21 | 0.64(58%) |
| 0.60 | 23 | 0.55(58%) |
| 0.60 | 25½ | 0.62(65%) |
| 0.56 | 24 | 0.53(60%) |

The 5 batches were combined, dissolved in a mixture of DCM and methanol and the resulting clear solution concentrated under reduced pressure to give 3, 2.87 g.

LCMS: Purity 99% Retention time 5.6 minutes MI:M+1=384

NMR (CDCl₃): 8.23 (m, 1H), 7.97 (s, b, 1H), 7.90 (d, J=8 Hz), 7.60-7.44 (m, 5H), 7.36 (d, J=8 Hz, 1H), 7.11 (m, 1H), 6.87 (m 2H), 5.61 (t, J=1 Hz), 5.19 (t, J=2 Hz, 1H), 4.89 (s, 2H), 4.17 (t, J=1 Hz, 2H), 3.90 (s, 2H).

NMR (d6-DMSO): 9.65 (s, 1H), 8.03 (dd, J=1 Hz, 7 Hz, 1H), 7.97 (d, J=8 Hz, 2H), 7.74 (d, J=7 Hz, 1H), 7.61 (m, 1H), 7.51 (m, 1H), 7.44 (d, J=8 Hz, 2H), 7.16 (d, J=8 Hz, 1H), 6.97 (m, 1H), 6.78 (dd, J=1 Hz, 8 Hz, 1H), 6.59 (m, 1H), 5.73 (s, 1H), 5.32 (s, 1H), 4.90 (s, 2H), 4.81 (s, 2H), 4.27 (s, 2H).

### Example 8

### N-(2-Aminophenyl)-4-{[1-{2-[(2-aminophenyl)amino]-2-oxoethyl}-4-methylene-3,4-dihydroisoquinolin-2(1H)-yl]methyl}benzamide

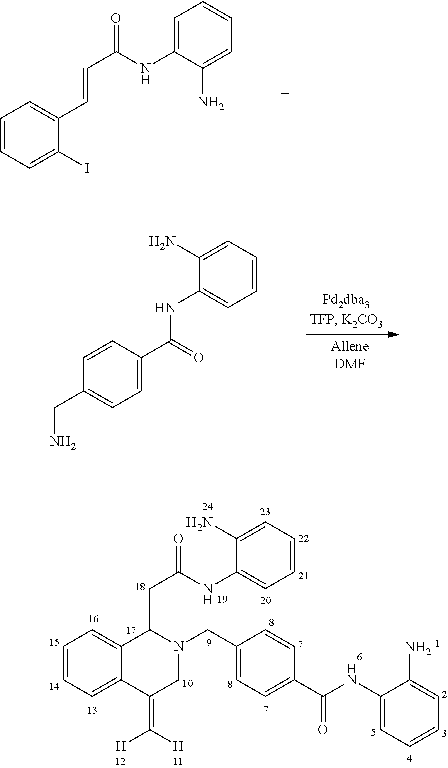 [SMILES: Nc1ccccc1NC(=O)/C=C/c1ccccc1I ; NCc1ccc(C(=O)Nc2ccccc2N)cc1 ; C=C1CN(Cc2ccc(C(=O)Nc3ccccc3N)cc2)C(CC(=O)Nc2ccccc2N)c2ccccc21]

Prepared by the general procedure from (2E)-N-(2-aminophenyl)-3-(2-iodophenyl)acrylamide (0.364 g, 1.0 mmol), 4-(aminomethyl)-N-(2-aminophenyl)benzamide (0.265 g, 1.1 mmol), Pd₂dba₃ (0.022 g, 0.025 mmol), TFP (0.023 g, 0.1 mmol), K₂CO₃ (0.276 g, 2.0 mmol) and allene (0.5 bar) in DMF (10 mL), heated at 80° C. for 24 h. Column chromatography (30 g silica, 1:3 v/v hexanes:EtOAc) afforded the product as a pale yellow solid. Crystallisation from DCM/hexane gave the product as pale yellow needles (0.208 g, 40%), m.p. 135-137° C.

(Found: C, 74.0; H, 6.05; N, 13.25. C₃₂H₃₁N₅O₂ requires: C, 74.25, H, 6.04, N, 13.53).

δH (CDCl₃, 500 MHz): 10.10 (s, 1H, N—H), 7.79 (s, b, 1H, N—H), 7.75-7.71 (m, 3H, 3×Ar—H), 7.37-7.29 (m, 5H, 5×Ar—H), 7.16 (d, J 6.8, 1H, Ar—H), 7.11-7.02 (m, 3H, 3×Ar—H), 6.87-6.78 (m, 4H, 4×Ar—H), 5.79 (s, 1H, H-12), 5.04 (s, 1H, H-11), 4.27 (dd, J 11.5, 2.8, 1H, H-17), 4.06 (d, J 15.4, 1H, H-10), 3.90 (s, b, 2H, H-1), 3.82 (d, J 13.0, 1H, H-9), 3.76 (d, J 13.0, 1H, H-9), 3.41 (d, J 15.4, 1H, H-10), 3.0 (dd, J 16.7, 11.5, 1H, H-18), 2.65 (dd, J 16.7, 2.8, 1H, H-18).

δC (CDCl₃, 75 MHz): 169.9, 166.0, 141.8, 141.6, 141.2, 135.7, 134.3, 131.9, 130.3, 129.5, 128.4, 128.0, 127.9, 127.7, 127.6, 125.8, 125.7, 124.8, 124.5, 124.1, 120.2, 119.6, 118.8, 118.3, 111.6, 60.0, 57.7, 49.4, 41.9.

v/max (film): 3381, 3225, 1644, 1616, 1495, 1454.

m/z (EZ) (%): 518 (MH⁺, 25), 146 (100).

### Example 9

### [2-(4-{[(2-Aminophenyl)amino]carbonyl}benzyl)-4-methylene-1,2,3,4-tetra hydroisoquinolin-1-yl]acetic acid

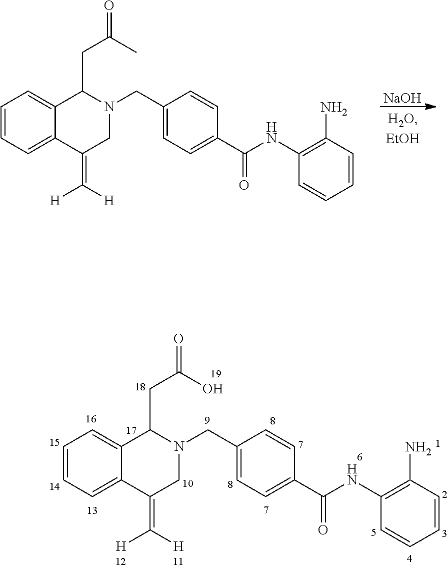 [SMILES: C=C1CN(Cc2ccc(C(=O)Nc3ccccc3N)cc2)C(CC(C)=O)c2ccccc21 ; "NaOH" ; C=C1CN(Cc2ccc(C(=O)Nc3ccccc3N)cc2)C(CC(=O)O)c2ccccc21]

To a stirred solution of methyl [2-(4-{[(2-aminophenyl)amino]carbonyl}benzyl)-4-methylene-1,2,3,4-tetrahydroisoquinolin-1-yl]acetate (0.115 g, 0.26 mmol) in EtOH (2 mL) was added NaOH (0.5 mL, 1M aq, 0.5 mmol). The mixture was stirred at room temperature for 18 h, and the organic solvent was removed in vacuo. HCl (10 mL, 1 M aq) was added, and the mixture was extracted with DCM (3×10 mL). The combined organic phases were dried (MgSO₄), filtered and concentrated in vacuo to give the product as colourless plates (0.069 g, 62%), m.p. >230° C.

Found: 450.1781; C₂₆H₂₅N₃O₃Na (MNa⁺) requires: 450.1788.

δ_{H} (500 MHz, DMSO-d₆): 9.66 (s, b, 1H, H-19), 7.95 (d, J 7.5, 2H, H-7), 7.79 (d, J 7.1, 1H, H-13), 7.41 (d, J 7.5, 2H, H-8), 7.36-7.31 (m, 2H, 2×Ar—H), 7.21-7.16 (m, 2H, 2×ArH), 6.99 (dd, J 7.7, 6.8, 1H, H-3), 6.81 (d, J 7.7, 1H, H-2), 6.62 (dd, J 6.8, 6.4, 1H, H-3), 5.82 (s, 1H, H-12), 5.01 (s, 1H, H-11), 4.31-4.26 (m, 1H, H-17), 3.88 (d, J 15.6, 1H, H-10), 3.74-3.67 (m, 2H, H-9), 3.25 (d, J 15.6, 1H, H-10), 2.84-2.78 (m, 1H, H-18), 2.62 (dd, J 15.4, 5.5, 1H, H-18).

δC (DMSO-d₆, 75 MHz): 172.7, 165.5, 143.5, 142.5, 136.6, 135.7, 133.8, 131.9, 128.8, 128.7, 128.1, 128.0, 127.3, 127.0, 126.8, 123.9, 123.7, 116.6, 116.5, 110.5, 60.1, 59.3, 57.0, 41.0.

v/max (film): 3412, 1641, 1502, 1451, 1315.

m/z (EZ) (%): 450 (MNa⁺, 100)

### Example 10

### N-(2-Aminophenyl)-4-{[1-{2-[(2-hydroxyethyl)amino]-2-oxoethyl}-4-methylene-3,4-dihydroisoquinolin-2(1H)-yl]methyl}-benzamide

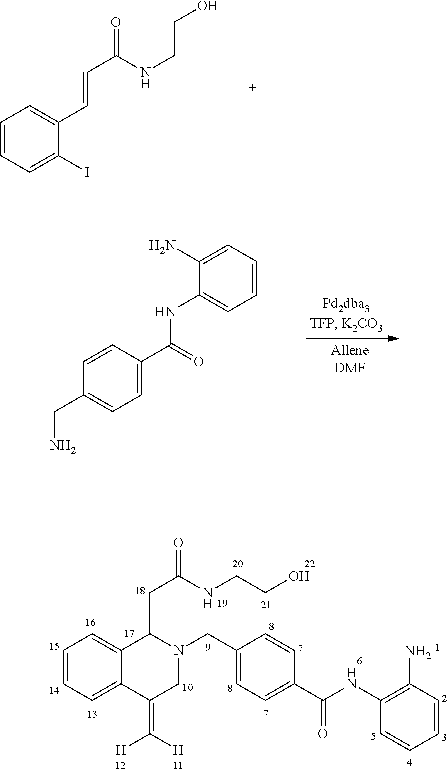 [SMILES: O=C(/C=C/c1ccccc1I)NCCO ; NCc1ccc(C(=O)Nc2ccccc2N)cc1 ; C=C1CN(Cc2ccc(C(=O)Nc3ccccc3N)cc2)C(CC(=O)NCCO)c2ccccc21]

Prepared by the general procedure from (2E)-N-(2-hydroxyethyl)-3-(2-iodophenyl)acrylamide (0.159 g, 0.5 mmol), 4-(aminomethyl)-N-(2-aminophenyl)benzamide (0.133 g, 0.55 mmol), Pd₂dba₃ (0.011 g, 0.0125 mmol), TFP (0.012 g, 0.05 mmol), K₂CO₃ (0.138 g, 1.0 mmol) and allene (0.5 bar) in DMF (10 mL), heated at 80° C. for 24 h. Column chromatography (30 g silica, EtOAc to 99:1 v/v EtOAc:MeOH) afforded the product as an amorphous pale yellow solid (0.108 g, 46%).

Found: 471.2388; C₂₈H₃₁N₄O₃ (MH⁺) requires: 471.2391.

δH (CDCl₃, 500 MHz): 8.17 (s, b, 2H, H-6, H-19), 7.90 (d, J 7.5, 2H, H-7), 7.71 (d, J 6.8, 1H, H-13), 7.42 (d, J 7.5, 2H, H-8), 7.35 (d, J 7.7, 1H, H-16), 7.31-7.28 (m, 2H, 2×Ar—H), 7.11-7.07 (m, 2H, 2×Ar—H), 6.86-6.82 (m, 2H, 2×Ar—H), 5.78 (s, 1H, H-12), 5.04 (s, 1H, H-11), 4.21 (dd, J 11.1, 3.0, 1H, H-17), 3.97 (d, J 15.2, 1H, H-10), 3.80 (d, J 12.8, 1H, H-9), 3.71 (d, J 15.2, 1H, H-10), 3.70-3.64 (m, 2H, H-21), 3.43-3.31 (m, 3H, H-20, H-9), 2.84 (dd, J 16.2, 11.1, 1H, H-18), 2.50 (dd, J 16.2, 3.0, 1H, H-18).

δ_{C} (75 MHz, CDCl₃): 172.5, 165.8, 142.2, 140.7, 135.7, 134.4, 133.7, 131.6, 129.7, 128.9, 128.0, 127.7, 127.3, 125.3, 124.6, 123.6, 119.7, 118.4, 110.6, 62.4, 59.4, 57.3, 49.5, 42.5, 41.6.

v/max (film): 3300, 3067, 2932, 1641, 1530.

m/z (EZ): (%) 471 (MH⁺, 100).

### Example 11

### N-(2-Aminophenyl)-4-{[4-methylene-1-[2-(4-methylpiperazin-1-yl)-2-oxoethyl]-3,4-dihydroisoquinolin-2(1H)-yl]methyl}benzamide

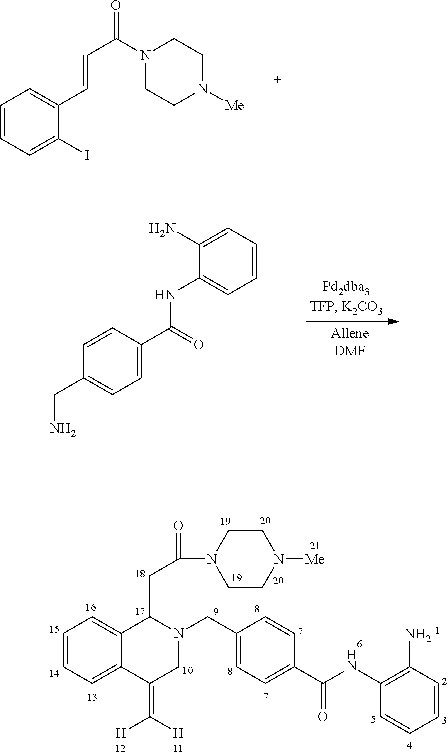 [SMILES: CN1CCN(C(=O)/C=C/c2ccccc2I)CC1 ; NCc1ccc(C(=O)Nc2ccccc2N)cc1 ; C=C1CN(Cc2ccc(C(=O)Nc3ccccc3N)cc2)C(CC(=O)N2CCN(C)CC2)c2ccccc21]

Prepared by the general procedure from 1-[(2E)-3-(2-iodophenyl)prop-2-enoyl]-4-methylpiperazine (0.356 g, 1.0 mmol), 4-(aminomethyl)-N-(2-aminophenyl)benzamide (0.265 g, 1.1 mmol), Pd₂dba₃ (0.022 g, 0.025 mmol), TFP (0.023 g, 0.1 mmol), K₂CO₃ (0.276 g, 2.0 mmol) and allene (0.5 bar) in DMF (10 mL), heated at 80° C. for 24 h. Column chromatography (30 g basic Al₂O₃, EtOAc) afforded the product as an amorphous pale pink solid (0.256 g, 50%).

Found: 510.2879; C₃₁H₃₆N₅O₂ (MH⁺) requires 510.2864.

δH (CDCl₃, 500 MHz): 8.05 (s, b, 1H, H-6), 7.84 (d, J 8.1, 2H, H-7), 7.68 (d, J 6.4, 1H, H-16), 7.41 (d, J 8.1, 2H, H-8), 7.32 (d, J 7.3, 1H, Ar—H), 7.26-7.23 (m, 2H, 2×Ar—H), 7.13 (d, J 6.6, 1H, Ar—H), 7.08 (t, J 7.7, 1H, Ar—H), 6.85-6.83 (m, 2H, 2×Ar—H), 5.70 (s, 1H, H-12), 4.95 (s, 1H, H-11), 4.38 (dd, J 6.0, 8.1, 1H, H-17), 3.91 (s, b, 2H, H-1), 3.87 (d, J 15.8, 1H, H-10), 3.75 (d, J 13.5, 1H, H-9), 3.77-3.74 (m, 1H, N—CH₂), 3.68 (d, J 13.5, 1H, H-9), 3.63-3.57 (m, 1H, N—CH₂), 3.42-3.37 (m, 1H, N—CH₂), 3.30 (d, J 15.8, 1H, H-10), 3.22-3.16 (m, 1H, N—CH₂), 2.96 (dd, J 14.1, 8.1, 1H, H-18), 2.67 (dd, J 14.1, 6.0, 1H, H-18). 2.41-2.33 (m, 2H, N—CH₂), 2.29-2.23 (m, 1H, N—CH₂), 2.26 (s, 3H, H-21), 2.19-2.13 (m, 1H, N—CH₂).

δC (CDCl₃, 75 MHz): 169.6, 165.9, 143.5, 140.8, 136.7, 135.9, 133.1, 132.1, 129.3, 128.4, 128.0, 127.3, 127.2, 127.1, 125.3, 124.7, 123.6, 119.7, 118.3, 109.8, 60.0, 57.7, 55.0, 54.7, 50.1, 46.0, 45.9, 41.7, 39.1.

v/max (film): 3410, 1625, 1503, 1450.

m/z (EZ) (%): 497 (MH⁺, 100).

### Example 12

### N-(2-Aminophenyl)-4-{[1-{2-[(2-{[2E)-3-(3-hydroxy-4-methoxyphenyl)-2-(3,4,5-trimethoxyphenyl)prop-2-enoyl]amino}ethyl)amino]-2-oxoethyl}-4-methylene-3,4-dihydroisoquinolin-2(1H)-yl]methyl}benzamide

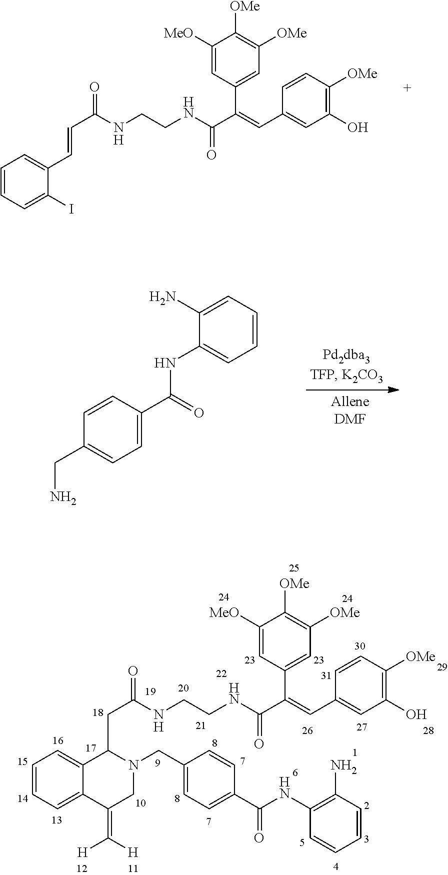 [SMILES: COc1ccc(/C=C(/C(=O)NCCNC(=O)/C=C/c2ccccc2I)c2cc(OC)c(OC)c(OC)c2)cc1O ; NCc1ccc(C(=O)Nc2ccccc2N)cc1 ; C=C1CN(Cc2ccc(C(=O)Nc3ccccc3N)cc2)C(CC(=O)NCCNC(=O)/C(=C/c2ccc(OC)c(O)c2)c2cc(OC)c(OC)c(OC)c2)c2ccccc21]

Prepared by the general procedure from (2E)-3-(3-hydroxy-4-methoxyphenyl)-N-(2-{[(2E)-3-(2-iodophenyl)prop-2-enoyl]amino}ethyl)-2-(3,4,5-trimethoxyphenyl)acrylamide (0.329 g, 0.5 mmol), 4-(aminomethyl)-N-(2-aminophenyl)benzamide (0.133 g, 0.55 mmol), Pd₂dba₃ (0.011 g, 0.0125 mmol), TFP (0.012 g, 0.05 mmol), K₂CO₃ (0.138 g, 1.0 mmol) and allene (0.5 bar) in DMF (10 mL), heated at 80° C. for 24 h. Column chromatography (25 g silica, EtOAc) afforded the product as a pale yellow solid. Crystallisation from DCM/hexane gave the product as pale yellow prisms (0.192 g, 47%), m.p. >230° C.

(Found: C, 69.50; H, 6.00; N, 8.40; C₄₇H₄₉N₅O₈ requires: C, 69.53; H, 6.08; N, 8.63%).

δH (CDCl₃, 500 MHz): 8.43 (s, b, 1H, H-6), 8.11 (d, J 8.1, 2H, H-7), 7.72 (d, J 8.6, 1H, H-13), 7.51 (s, 1H, H-26), 7.39-7.33 (m, 3H, 3×Ar—H), 7.31-7.28 (m, 2H, 2×Ar—H), 7.10-7.05 (m, 2H, H-3, H-5), 6.86 (d, J 7.3, 1H, H-2), 6.75 (t, J 7.3, 1H, H-4), 6.62 (d, J 8.5, 1H, H-30), 6.43 (s, 1H, H-27), 6.40-6.35 (m, 3H, H-31, H-23), 5.92 (s, b, 1H, N—H), 5.78 (s, 1H, H-12), 5.47 (s, b, 1H, N—H), 5.11 (s, 1H, H-11), 4.17 (s, b, 1H, H-28), 4.11 (d, J 15.6, 1H, H-10), 4.02 (dd, J 11.8, 2.3, 1H, H-17), 3.92 (s, 3H, H-25), 3.85-3.77 (m, 5H, 2×H-1, 1×H-9, 2×H-21), 3.83 (s, 3H, H-29), 3.77 (s, 6H, H-24), 3.57 (d, J 12.8, 1H, H-9), 3.48 (d, J 15.6, 1H, H-10), 3.17-3.08 (m, 2H, H-20), 2.78 (dd, J 17.1, 11.8, 1H, H-18), 2.36 (dd, J 17.1, 2.3, 1H, H-18).

δC (CDCl₃, 75 MHz): 171.6, 168.3, 166.0, 154.4, 147.4, 145.1, 142.6, 141.8, 141.3, 137.6, 135.6, 134.3, 134.1, 131.6, 131.0, 129.5, 129.0, 128.6, 128.2, 127.8, 127.4, 127.1, 125.9, 124.8, 123.6, 122.4, 119.3, 118.2, 116.5, 110.6, 110.1, 106.7, 61.1, 57.9, 57.4, 56.4, 55.8, 51.0, 41.0, 39.5, 38.2.

v/max (film): 3419, 1644, 1508.

m/z (EZ) (%): 812 (MH⁺, 100).

### Example 13

### Methyl 2-(4-{[(2-aminophenyl)amino]carbonyl}benzyl)-4-methylene-1-oxo-1,2,3,4-tetrahydroisoquinoline-6-carboxylate

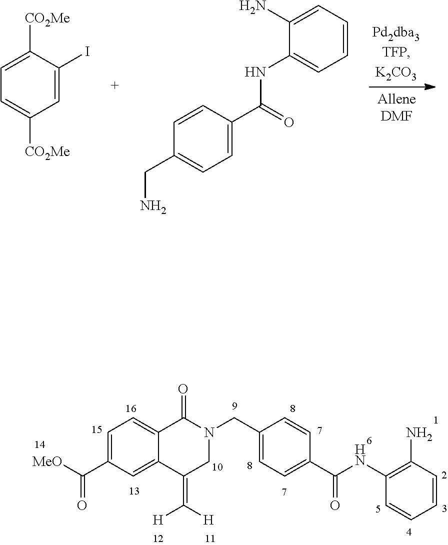 [SMILES: COC(=O)c1ccc(C(=O)OC)c(I)c1 ; NCc1ccc(C(=O)Nc2ccccc2N)cc1 ; C=C1CN(Cc2ccc(C(=O)Nc3ccccc3N)cc2)C(=O)c2ccc(C(=O)OC)cc21]

Prepared by the general procedure from dimethyl 2-iodoterephthalate (0.160 g, 0.5 mmol), 4-(aminomethyl)-N-(2-aminophenyl)benzamide (0.133 g, 0.55 mmol), Pd₂dba₃ (0.011 g, 0.0125 mmol), TFP (0.012 g, 0.05 mmol), K₂CO₃ (0.138 g, 1.0 mmol) and allene (0.5 bar) in DMF (10 mL), heated at 80° C. for 24 h. Column chromatography (25 g silica, Et₂O) afforded the product as a pale orange solid. Crystallisation from toluene gave the product as colourless needles (0.150 g, 68%), m.p. 196-198° C.

Found: 442.1760; C₂₆H₂₄N₃O₄ (MH⁺) requires 442.1761.

δH (CDCl₃, 500 MHz): 8.27 (d, J 8.2, 1H, H-16), 8.23 (d, J 1.2, 1H, H-13), 8.08 (dd, J 8.2, 1.2, 1H, H-15), 7.90-7.88 (m, 2H, H-6 & H-7), 7.44 (d, J 8.0, 2H, H-8), 7.33 (d, J 7.7, 1H, H-5), 7.11-7.08 (m, 1H, H-4), 6.86-6.84 (m, 2H, H-2 & H-3), 5.71 (s, 1H, H-12), 5.25 (s, 1H, H-11), 4.88 (s, 2H, H-9), 4.18 (s, 1H, H-10), 3.97 (s, 3H, H-14), 3.87 (s, b, 2H, H-1).

δC (CDCl₃, 75 MHz): 166.25, 162.6, 140.8, 140.6, 135.9, 135.4, 133.7, 133.5, 130.1, 129.6, 129.1, 128.3, 127.9, 127.3, 125.1, 124.8, 124.6, 119.9, 118.5, 113.9, 52.5, 51.9, 50.2.

v/max (film): 1722, 1652, 1630, 1487, 1271.

m/z (EZ) (%): 442 (MH⁺, 100), 464 (MNa⁺, 80).

### Example 14

### N-(2-Aminoethyl)-2-(4-{[(2-aminophenyl)amino]carbonyl}benzyl)-4-methylene-1-oxo-1,2,3,4-tetrahydroisoquinoline-6-carboxamide

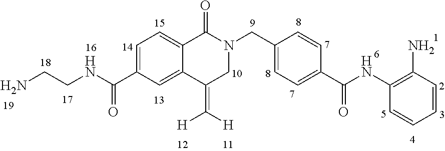 [SMILES: C=C1CN(Cc2ccc(C(=O)Nc3ccccc3N)cc2)C(=O)c2ccc(C(=O)NCCN)cc21]

Methyl 2-(4-{[(2-aminophenyl)amino]carbonyl}benzyl)-4-methylene-1-oxo-1,2,3,4-tetra hydroisoquinoline-6-carboxylate (0.429 g, 1 mmol) was dissolved in EtOH (5 mL) and 1M aq NaOH (2.5 mL, 2.5 mmol) and stirred at room temperature for 6 h. The organic solvent was removed in vacuo, and the resulting mixture acidified to pH 4 with HCl (1M aq). The product was collected by filtration, dried in vacuo and used in the next step without further purification.

To a stirred solution of the resulting solid material (0.427 g, 1 mmol) and 4-(4,6-dimethoxy-1,3,5-triazin-2-yl)-4-methylmorpholin-4-ium chloride (0.276 g, 1 mmol) in DMF (5 mL) was added tert-butyl (2-aminoethyl)carbamate (0.224 g, 1.4 mmol) in one portion at room temperature. After stirring at room temperature for 16 h, the mixture was diluted with water (50 mL) and extracted with EtOAc (3×20 mL). The combined organic extracts were washed with water (20 mL), dried (MgSO₄), filtered and concentrated in vacuo. Flash chromatography (25 g SiO₂, 4:1 v/v EtOAc:hexanes) gave a colourless solid (0.372 g, 65%) which was used without further purification.

To a stirred solution of the resulting solid (0.569 g, 1 mmol) in DCM (5 mL) was added TFA (0.150 g, 2 mmol) in one portion at room temperature. After stirring for 2 h, the mixture was diluted with DCM (10 mL), washed with 1M aq NaOH (2×10 mL), dried (MgSO₄), filtered and concentrated in vacuo. Trituration with Et₂O gave the product as pale yellow plates (0.415 g, 88%), m.p. 172-174° C. (dec).

Found: 470.2189; C₂₇H₂₈N₅O₃ requires 470.2187.

δH (DMSO-d₆, 500 MHz): 8.18 (s, b, 2H, H-13, H-6), 8.11 (d, J 8.1, 1H, H-15), 7.99 (d, J 8.1, 2H, H-7), 7.96 (d, J 8.1, 1, H-14), 7.44 (d, J 8.1, 2H, H-8), 7.21 (q, J 3.0, 1H, H-16), 7.17 (d, J 7.5, 1H, H-5), 6.98 (dd, J 8.1, 7.3, 1H, H-3), 6.79 (d, J 8.1, 1H, H-2), 6.61 (dd, J 7.5, 7.3, 1H, H-4), 5.83 (s, 1H, H-12), 5.41 (s, 1H, H-11), 4.92 (s, b, 2H, H-19), 4.82 (s, 2H, H-9), 4.31 (s, 2H, H-10), 3.36-3.32 (m, 2H, H-18), 2.81-2.79 (m, 2H, H-17).

δC (DMSO-d₆, 75 MHz): 166.4, 165.4, 162.2, 143.0, 141.0, 137.7, 136.1, 136.0, 134.0, 129.6, 128.5, 128.0, 127.9, 127.0, 126.9, 123.9, 123.0, 116.9, 116.7, 115.5, 114.3, 51.7, 49.7, 37.6, 34.6.

v/max (film): 3030, 1635, 1531, 1503.

m/z (%): 470 (MH⁺, 100), 492 (MNa⁺, 10).

### Example 15

### N-(2-Aminophenyl)-4-{[1-(cyanomethyl-4-methylene-3,4-dihydroisoquinolin-2(1H)-yl]sulfonyl}benzamide

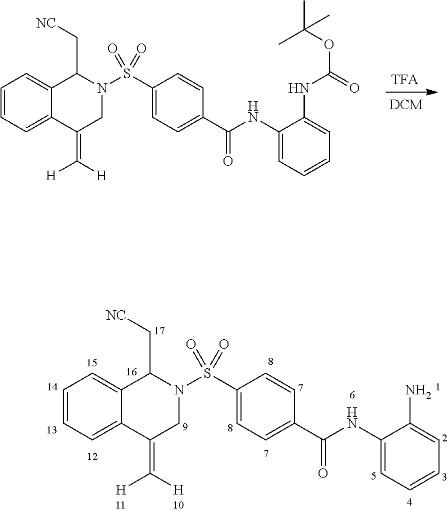 [SMILES: C=C1CN(S(=O)(=O)c2ccc(C(=O)Nc3ccccc3NC(=O)OC(C)(C)C)cc2)C(CC#N)c2ccccc21 ; C=C1CN(S(=O)(=O)c2ccc(C(=O)Nc3ccccc3N)cc2)C(CC#N)c2ccccc21]

To a stirred solution of tert-butyl{2-[(4-{[1-(cyanomethyl)-4-methylene-3,4-dihydroisoquinolin-2(1H)-yl]sulfonyl}benzoyl)amino]phenyl}carbamate (0.279 g, 0.5 mmol) in DCM (5 mL) was added TFA (0.11 g, 1.0 mmol) in one portion at room temperature. The resulting mixture was stirred at room temperature for 4 hours, washed with NaOH aq., 2×5 mL), dried (MgSO₄), filtered and concentrated in vacuo. Column chromatography (25 g SiO₂, Et₂O) gave the product as pale yellow prisms (0.153 g, 67%), m.p. 213-215° C.

Found: 459.1488; C₂₅H₂₃N₄O₃S requires 459.1485.

δH (CDCl₃, 500 MHz): 9.74 (s, 1H, H-6), 7.92 (d, J 8.3, 2H, H-7), 7.78 (d, J 8.3, 2H, H-8), 7.51 (d, J 7.9, 1H, H-12), 7.39 (d, J 7.7, 1H, H-15), 7.26 (dd, J 7.9, 7.5, 1H, H-13), 7.17 (dd, J 7.7, 7.5, 1H, H-14), 7.12 (d, J 7.5, 1H, H-5), 6.98 (dd, J 7.5, 7.3, 1H, H-3), 6.78 (d, J 7.5, 1H, H-2), 6.60 (dd, J 7.5, 7.3, 1H, H-4), 5.58 (s, 1H, H-11), 5.52 (dd, J 9.8, 4.3, 1H, H-16), 5.19 (s, 1H, H-10), 4.55 (d, J 16.7, 1H, H-9), 4.41 (d, J 16.7, 1H, H-9), 3.38 (dd, J 17.3, 9.8, 1H, H-17), 3.11 (dd, J 17.3, 4.3, 1H, H-17).

δC (CDCl₃, 75 MHz): 164.1, 143.5, 141.5, 138.5, 134.3, 132.4, 130.9, 128.6, 128.4, 128.1, 127.7, 127.5, 127.2, 124.2, 123.1, 118.4, 116.5, 116.4, 111.4, 53.2, 45.0, 24.9.

v/max (film) 2985, 1663, 1624, 1501.

m/z (EZ) (%) 459 (MH⁺, 55), 481 (MNa⁺, 100).

### Example 16

### In Vitro Assay for HDAC 3 and HDAC 6 Inhibitory Activity

The activity of the compounds of the invention against HDAC 3 (Class I) and HDAC 6 (Class II) can be determined using the Caliper Life Sciences HDAC inhibitor profiling assay, which utilises microfluidic mobility-shift detection.

### Reagents:

Reaction buffer: 25 mM Tris/Cl, pH 8.0, 137 mM NaCl, 2.7 mM KCl, 1 mM MgCl₂, 0.1 mg/ml BSA (fatty acid free).

### Stop Buffer: 100 mM HEPES, pH 7.5, 10 mM EDTA, 0.04% Brij-35, 0.25% CR-3, and 2.5 μM Trichostatin A.

### Assay Protocol:

Stopped reactions were assembled in microtiter plate wells by adding 1 μL of the inhibitor compound in DMSO, 15 μL 2× Enzyme, and 15 μL 2× peptide. The reactions were incubated for 1 hour at room temperature, stopped with the addition of 45 μL Stop Solution, and read on the LabChip EZ Reader®. The curves were fit with sigmoidal dose response algorithm using GraphPad Prism.

Although the pharmacological properties of the compounds of the formula I will vary with structural change as expected, compounds of the formula I, were found to be active in the above screens against HDAC 3 (a Class I HDAC) and relatively inactive against HDAC 6 (a Class II HDAC). In general, the activity possessed by compounds of the formula I, is represented by an IC₅₀ of less than 10 μM against HDAC 3. Preferred compounds of the invention have an IC₅₀ of less than 5 μM against HDAC 3, and the most preferred compounds of the invention have an IC₅₀ of less than 1 μM. With regard to HDAC 6 activity, the compounds of the invention, in general, demonstrate an IC₅₀ value which is greater than 10 μM.

The inhibitory activity of selected compounds of the invention against HDAC 3 and HDAC 6 is shown in Table 1 Below.

TABLE 1

| | |
|---|---|
| Compound/Example | IC50 (μM) |

| number | HDAC3 | HDAC6 |
|---|---|---|
| 1 | 0.89 | >10 |
| 2 | 0.51 | >10 |
| 3 | 0.98 | >10 |
| 4 | 0.26 | >10 |
| 5 | 2.7 | >10 |
| 6 | 0.31 | >10 |
| 7 | 0.22 | >10 |
| 8 | 0.35 | >10 |
| 9 | 0.78 | >10 |
| 10 | 0.67 | >10 |
| 11 | 0.56 | >10 |
| 13 | 0.27 | >10 |
| 14 | 0.23 | >10 |
| 15 | 1.4 | >10 |

It will be understood, the IC₅₀ values quoted above are not absolute and further measurements of the IC₅₀ value for a compound may result in a different geometric mean IC₅₀ value.

### Example 17

### In Vitro Assay for Class I and Class II HDACs

The inhibitory effects of the compounds of Examples 7 and 9 above on HDAC activity were determined using a fluorescence-based assay with electrophoretic separation of substrate and product carried out using a microfluidic system followed by quantitation of fluorescence intensity in the substrate and product peaks.

The assays were performed using isolated HDAC isoforms that had been expressed as 6×His-tagged fusion proteins in a baculovirus expression system in Sf9 cells. HDACs 1, 2, 3, 6, and 8 were expressed as full length fusion proteins. For HDAC-4 and 7 only catalytic domains were expressed (HDAC-4 CAT and HDAC-7 CAT). The HDAC10 fusion protein was expressed as a carboxy-terminal deletion of 38 amino acids (residues 632-669). HDAC3 was coexpressed with a fragment of the SMRT gene (residues 395-489) to generate enzymatically active protein. Purified proteins were incubated with 1 μM carboxyfluorescein (FAM)-labeled acetylated peptide substrate and test compound for 17 h at 25° C. in HDAC assay buffer containing 100 mM HEPES (pH 7.5), 25 mM KCl, 0.1% BSA, and 0.01% Triton X-100. Reactions were terminated by the addition of buffer containing 0.078% SDS for a final SDS concentration of 0.05%. Substrate and product were separated electrophoretically using a Caliper LabChip 3000 system with blue laser excitation and green fluorescence detection (CCD2). The fluorescence intensity in the substrate and product peaks was determined using the Well Analyzer software on the Caliper system. The reactions were performed in duplicate for each sample.

IC50 values were automatically calculated using the IDBS XLFit version 4.2.1 plug-in for Microsoft Excel and the XLFit 4 Parameter Logistic Model (sigmoidal dose-response model): (A+((B−A)/1+(C/x)^{D})), where x is compound concentration, A is the estimated minimum, B is the estimated maximum of % inhibition, C is the inflection point, and D is the Hill slope of the sigmoidal curve. The standard errors of the IC50s were automatically calculated using the IDBS XLFit version 4.2.1 plug-in for Microsoft Excel and the formula xf4_FitResultStdError( ).

The pharmacological properties of the compounds of the formula I will vary with structural change, as expected. In general, the compounds of the formula I, were found to possess good activity in the above screen against the HDAC 2 and HDAC 3 (both of which are Class I HDACs) and were relatively less active against HDACs 1, 4, 6, 7, 8, and 10. In general activity possessed by compounds of the formula I possess an IC₅₀ of less than 10 μM against HDAC 2 and/or 3 in the above assay (preferred compounds have an IC₅₀ of less than 1 μM, more preferably less than 0.1 μM). The compounds of the invention may also exhibit an IC₅₀ of greater than 10 μM against HDACs 6 and 8, and greater than 5 μM against the class II HDACs 4, 7 and 10.

Particular data obtained for the compounds of Examples 7 and 9 are shown below.

| | CLASS 1 | | | | CLASS 2 | | | |
|---|---|---|---|---|---|---|---|---|
| | HDAC 1 | HDAC 2 | HDAC 3 | HDAC 8 | HDAC 4-CAT | HDAC 6 | HDAC 7-CAT | HDAC 10 |
| Compound | IC50 | IC50 | IC50 | IC50 | IC50 | IC50 | IC50 | IC50 |
| number | (μM) | (μM) | (μM) | (μM) | (μM) | (μM) | (μM) | (μM) |
| 7 | 8.7 | 0.0544 | 0.0287 | >30 | 9.22 | >30 | 7.53 | 8.04 |
| 9 | >30 | 2.11 | 0.797 | >30 | >30 | >30 | >30 | >30 |

### Example 18

### Evaluation of the Activity of the Compound of Example 7 (MI-192) in MTS, CD11b and CYP26 Assays

### Experimental Procedures

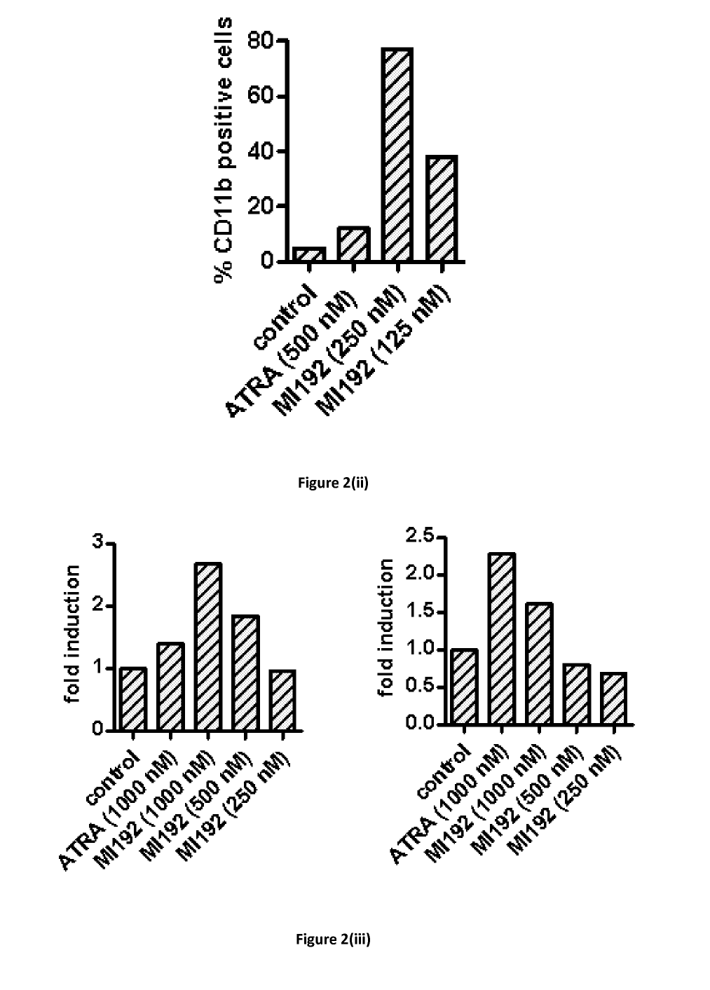

MTS Assays (FIG. 2i)

HL60 or NB4 cells were diluted in complete growth medium and plated at a density of 5000 cells per 0.1 ml in wells of a 96 well plate. Cells were then treated with various concentrations of the compound of Example 7 (MI-192) or were left untreated as a control in a total volume of 0.2 ml of complete growth medium. After 3 days, relative cell numbers were determined using the MTS assay, as described by the manufacturer (Promega, Southampton, UK). The results shown in FIG. 2(i) are absorbance (490 nm) relative to control cells (untreated cells 100%).

CD11b (FIG. 2ii)

HL60 cells were cultured at 0.5×10⁶ cells/ml in complete growth medium. Cells (2 ml) were then treated with ATRA (sourced commercially), the compound of Example 7 (MI-192), or left untreated as controls. After 3 days, CD11b expression was determined by flow cytometry using a PE-conjugated anti-CD11b monoclonal antibody.

CYP26 (FIG. 2iii)

HL60 or NB4 cells were cultured at 1.0×10⁶ cells/ml in complete growth medium. Cells (10 ml) were then treated with ATRA, the compound of Example 7(MI-192), or left untreated as controls. After 1 day, the expression of CYP26 and BACT RNAs were determined using quantitative PCR (Taqman). The expression of CYP26 RNA was normalised to BACT, and expression in control cells was set to 1.0.

As can be seen from the data shown in FIG. 2, the compound of Example 7 (MI-192) induces expression of CYP26, a gene that is repressed by HDAC3¹². (Note that CYP26 is a specialised CYP important for retinoic acid metabolism but not a major player in drug metabolism). The compound of Example 7 also promotes growth inhibition and differentiation in AML cells (FIG. 2). Interestingly, in HL60 cells the transcription modulating activity of MI-192 exceeded that of ATRA, the “gold standard” differentiation agent for AML.

### REFERENCES

1. Witt O, Deubzer H E, Milde T, Oehme I. HDAC family: What are the cancer relevant targets? Cancer Lett. 2008.
2. Karagianni P, Wong J. HDAC3: taking the SMRT-N-CoRrect road to repression. Oncogene. 2007; 26:5439-5449.
3. Zhang J, Kalkum M, Chait B T, Roeder R G. The N-CoR-HDAC3 nuclear receptor corepressor complex inhibits the JNK pathway through the integral subunit GPS2. Mol. Cell. 2002; 9:611-623.
4. Guenther M G, Lane W S, Fischle W, Verdin E, Lazar M A, Shiekhattar R. A core SMRT corepressor complex containing HDAC3 and TBL1, a WD40-repeat protein linked to deafness. Genes Dev. 2000; 14:1048-1057.
5. Petrie K, Zelent A, Waxman S. Differentiation therapy of acute myeloid leukemia: past, present and future. Curr Opin Hematol. 2009; 16:84-91.
6. Minucci S, Nervi C, Lo Coco F, Pelicci P G. Histone deacetylases: a common molecular target for differentiation treatment of acute myeloid leukemias? Oncogene. 2001; 20:3110-3115.
7. Grignani F, De Matteis S, Nervi C, et al. Fusion proteins of the retinoic acid receptor-alpha recruit histone deacetylase in promyelocytic leukaemia. Nature. 1998; 391:815-818.
8. Lutterbach B, Hou Y, Durst K L, Hiebert S W. The inv(16) encodes an acute myeloid leukemia 1 transcriptional corepressor. Proc Natl Aced Sci USA. 1999; 96:12822-12827.
9. Lutterbach B, Westendorf J J, Linggi B, et al. ETO, a target of t(8; 21) in acute leukemia, interacts with the N-CoR and mSin3 corepressors. Mol Cell Biol. 1998; 18:7176-7184.
10. Gelmetti V, Zhang J, Fanelli M, Minucci S, Pelicci P G, Lazar M A. Aberrant recruitment of the nuclear receptor corepressor-histone deacetylase complex by the acute myeloid leukemia fusion partner ETO. Mol Cell Biol. 1998; 18:7185-7191.
11. Redner R L, Liu J M. Leukemia fusion proteins and co-repressor complexes: changing paradigms. J Cell Biochem. 2005; 94:864-869.
12. Atsumi A, Tomita A, Kiyoi H, Naoe T. Histone deacetylase 3 (HDAC3) is recruited to target promoters by PML-RARalpha as a component of the N-CoR co-repressor complex to repress transcription in vivo. Biochem Biophys Res Commun. 2006; 345:1471-1480.
13. Ozeki M, Shively J E. Differential cell fates induced by all-trans retinoic acid-treated HL-60 human leukemia cells. J Leukoc Biol. 2008; 84:769-779.
14. Cote S, Rosenauer A, Bianchini A, et al. Response to histone deacetylase inhibition of novel PML/RARalpha mutants detected in retinoic acid-resistant APL cells. Blood. 2002; 100:2586-2596.
15. Stimson L, Wood V, Khan O, Fotheringham S, La Thangue N B. HDAC inhibitor-based therapies and haematological malignancy. Ann Oncol. 2009; 20:1293-1302.
16. Hayashi A, Horiuchi A, Kikuchi N, et al. Type-specific roles of histone deacetylase (HDAC) overexpression in ovarian carcinoma: HDAC1 enhances cell proliferation and HDAC3 stimulates cell migration with down-regulation of E-cadherin. Int J. Cancer.
17. Khabele D, Son D S, Parl A K, et al. Drug-induced inactivation or gene silencing of class I histone deacetylases suppresses ovarian cancer cell growth: implications for therapy. Cancer Biol Ther. 2007; 6:795-801.
18. Wilson A J, Byun D S, Popova N, et al. Histone deacetylase 3 (HDAC3) and other class I HDACs regulate colon cell maturation and p21 expression and are deregulated in human colon cancer. J Biol. Chem. 2006; 281:13548-13558.
19. Thangaraju M, Carswell K N, Prasad P D, Ganaphthy V. Colon cancer cells maintain low levels of pyruvate to avoid cell death caused by inhibition of HDAC1/HDAC3. Biochem J. 2009; 417:379-389.
20. Spurling C C, Godman C A, Noonan E J, Rasmussen T P, Rosenberg D W, Giardina C. HDAC3 overexpression and colon cancer cell proliferation and differentiation. Mol. Carcinog. 2008; 47:137-147.
21. Glaser K B, Li J, Stayer M J, Wei R Q, Albert D H, Davidsen S K. Role of class I and class II histone deacetylases in carcinoma cells using siRNA. Biochem Biophys Res Commun. 2003; 310:529-536.
22. Nguyen A, Su L, Campbell B, Poulin N M, Nielsen T O. Synergism of heat shock protein 90 and histone deacetylase inhibitors in synovial sarcoma. Sarcoma. 2009; 2009:794901.

What is claimed is:

1. A compound of the formula (I): 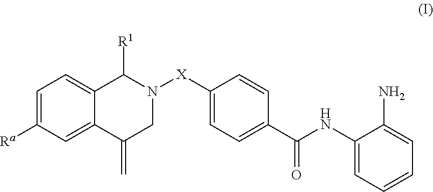
R^{a} is independently selected from hydrogen, —C(O)O—R^{b} or —C(O)NHR^{b}, where R^{b} is hydrogen or a C₁₋₆ alkyl group which is optionally substituted by one or more substituents independently selected from halogen, cyano, amino, hydroxy and C₁₋₂alkoxy;
X is —C(O)—, —S—, —S(O)—, —S(O)₂— or a C₁₋₂ alkylene linker;
R¹ is selected from hydrogen, oxo, C₁₋₂alkyl optionally substituted by halo, nitro or cyano;
or R¹ is a group:
-Q¹-X¹—R²
wherein:
Q¹ is a Cl_{—}2alkylene;
X¹ is selected from —O—, —N(R^{x})—, —C(O)—, —C(O)—O—, —O—C(O)—, —C(O)N(R^{x})—, —N(R^{x})C(O)—;
R^{x} is selected from hydrogen or C₁₋₂alkyl;
R² is selected from hydrogen, C₁₋₄alkyl, phenyl, wherein any C₁₋₄alkyl or phenyl group is optionally substituted with one or more substituents selected from halogen, cyano, amino, hydroxyl, C₁₋₂alkyl, C₁₋₂alkoxy or a group:
X²—R³
where X² is selected from —O—, —NH—, —C(O)—, —C(O)—O—, —O—C(O)—, —C(O)NH—, —NHC(O)—;
R³ is C₁₋₄alkyl or C₂₋₄alkenyl which is optionally substituted by halo, hydroxy, amino, cyano, C₁₋₂alkyl, C₁₋₂alkoxy, or a phenyl group which is optionally further substituted by one or more substituent groups selected from hydroxy, halo, cyano, nitro or methoxy;
or R^{x} and R² are linked such that, together with the nitrogen atom to which they are attached, they form a piperidine, piperazine, N-methyl piperazine or morpholino ring;
or a pharmaceutically acceptable salt or solvate thereof.

2. A compound according to claim 1, wherein R^{a} is independently selected from hydrogen, —C(O)O—R^{b} or —C(O)NHR^{b}, where R^{b} is hydrogen or a C₁₋₄alkyl group which is optionally substituted by one or more substituents independently selected from amino, hydroxy and C₁₋₂alkoxy.

3. A compound according to claim 2, wherein R^{a} is hydrogen, —C(O)O—CH₃, or —C(O)NH—CH₂—CH₂—NH₂.

4. A compound according to claim 1, wherein X is selected from S(O)₂ and methylene.

5. A compound according to claim 4, wherein X is methylene.

6. A compound according to claim 1, wherein R¹ is selected from hydrogen, oxo, C₁₋₂alkyl optionally substituted by cyano; or R¹ is a group:
-Q¹-X¹—R²
wherein:
Q¹ is methylene;
X¹ is selected from —C(O)—O—, or —C(O)N(R^{x})—;
R^{x} is hydrogen;
R² is selected from hydrogen, C₁₋₂alkyl, phenyl, wherein any C₁₋₂alkyl or phenyl group is optionally substituted with one or more substituents selected from halogen, cyano, amino, hydroxyl, or a group:
X²—R³
where:
X² is selected from —C(O)—O— or —C(O)NH—;
R³ is C₁₋₄alkyl or C₂₋₄alkenyl which is optionally substituted by halo, hydroxy, amino, C₁₋₂alkyl, C₁₋₂alkoxy, or a phenyl group which is optionally further substituted by one or more substituent groups selected from hydroxy or methoxy;
or R^{x} and R² are linked such that, together with the nitrogen atom to which they are attached, they form a piperidine, piperazine, N-methyl piperazine or morpholino ring;

7. A compound according to claim 6, wherein R¹ is oxo or methyl optionally substituted with cyano.

8. A compound according to claim 1, wherein said compound is selected from any one of the following: 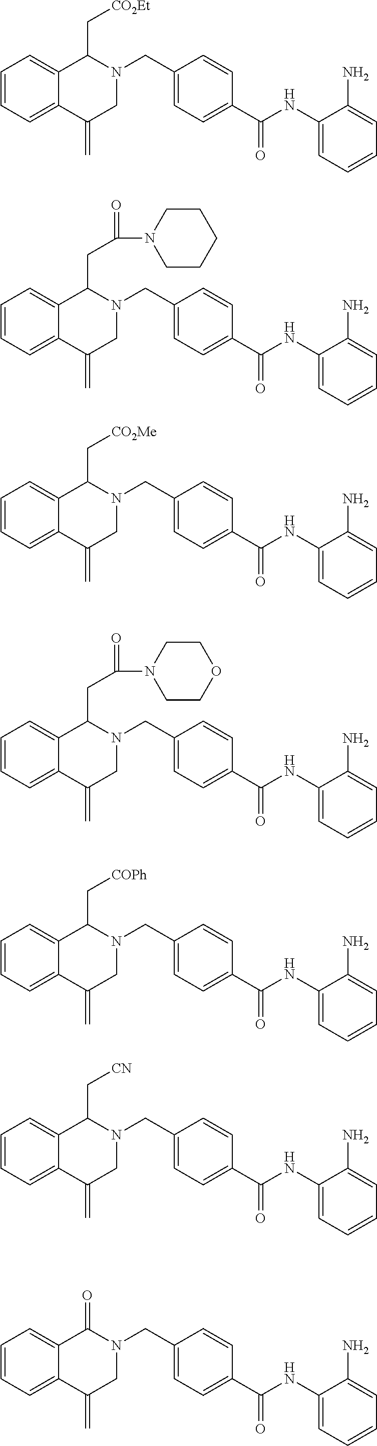 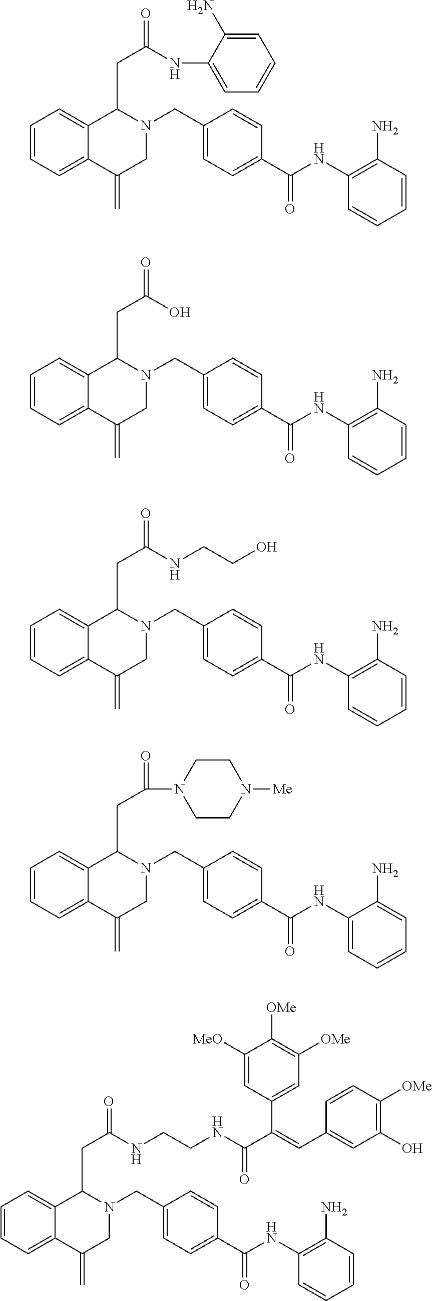 [SMILES: C=C1CN(Cc2ccc(C(=O)Nc3ccccc3N)cc2)C(CC(=O)Nc2ccccc2N)c2ccccc21 ; C=C1CN(Cc2ccc(C(=O)Nc3ccccc3N)cc2)C(CC(=O)O)c2ccccc21 ; C=C1CN(Cc2ccc(C(=O)Nc3ccccc3N)cc2)C(CC(=O)NCCO)c2ccccc21 ; C=C1CN(Cc2ccc(C(=O)Nc3ccccc3N)cc2)C(CC(=O)N2CCN(C)CC2)c2ccccc21 ; C=C1CN(Cc2ccc(C(=O)Nc3ccccc3N)cc2)C(CC(=O)NCCNC(=O)/C(=C/c2ccc(OC)c(O)c2)c2cc(OC)c(OC)c(OC)c2)c2ccccc21] 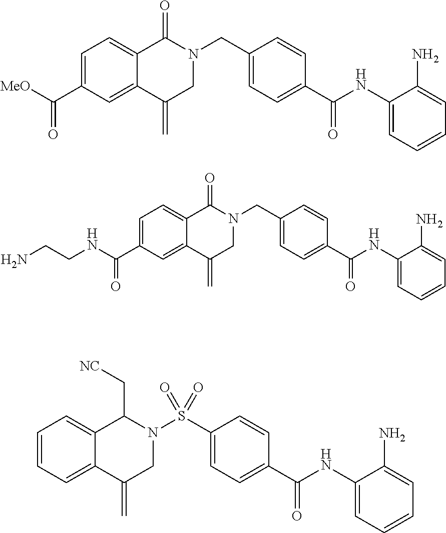 [SMILES: C=C1CN(Cc2ccc(C(=O)Nc3ccccc3N)cc2)C(=O)c2ccc(C(=O)OC)cc21 ; C=C1CN(Cc2ccc(C(=O)Nc3ccccc3N)cc2)C(=O)c2ccc(C(=O)NCCN)cc21 ; C=C1CN(S(=O)(=O)c2ccc(C(=O)Nc3ccccc3N)cc2)C(CC#N)c2ccccc21]
or a pharmaceutically acceptable salt or solvate thereof.

9. A pharmaceutical formulation comprising a compound according to claim 1 or a pharmaceutically acceptable salt or solvate thereof and a pharmaceutically acceptable carrier or excipient.

10.-13. (canceled)

14. A method for the treatment, prevention or delay of progression of cancer or inflammation in a subject, which comprises administering a therapeutically effective amount of a compound according to any one of claim 1 or a pharmaceutically acceptable salt or solvate thereof, or a pharmaceutical composition according to claim 9.

15. The use of a MTS assay, a CD11b expression assay and/or a CYP26 expression assay in HL60 or NB4 cells to assess the HDAC3 inhibitory activity of a compound as claimed in claim 1.
